(19) United States
(12) Patent Application Publication
(10) Pub. No.: US 2026/0284332 A1
(43) Pub. Date: Sep. 24, 2026

(54) SURGICAL HUMIDIFICATION SYSTEM

(71) Applicant: Fisher & Paykel Healthcare Limited, Auckland (NZ)

(72) Inventors: Christopher Thomas COLQUHOUN, Auckland (NZ); Rory MCKAY, Auckland (NZ); Zainab Ali A ALMADAN, Auckland (NZ); Thomas Heinrich BARNES, Surrey (GB); Christian Francis FISCHER, Auckland (NZ); Bernard Tsz Lun IP, Auckland (NZ); Eu-Lee TEH, Auckland (NZ); Manu PROSSER, Munich (DE); Joseph Patrick Walter STREVENS, Auckland (NZ); Lotte Gertrudis Theodora VAN DEN HEUIJ, Auckland (NZ); Qun SHAO, Auckland (NZ)

(21) Appl. No.: 19/484,541

(22) PCT Filed: May 16, 2024
(86) PCT No.: PCT/IB2024/054748

§ 371 Date: Nov. 14, 2025

Related U.S. Application Data
(60) Provisional application No. 63/502,566, filed on May 16, 2023.

(51) Int. Cl. A61M 13/00; A61F 7/00; A61F 7/12; A61M 11/04; A61M 39/22

(52) U.S. Cl. CPC A61M 13/006; A61F 7/0085; A61M 11/042; A61M 39/22; A61F 2007/0055; A61F 2007/0072; A61F 2007/126; A61M 2202/0225; A61M 2205/3334; A61M 2205/3344; A61M 2205/3368; A61M 2205/75

(57) ABSTRACT

Disclosed is a surgical humidification system, comprising: a humidifier configured to heat and humidify a gases flow to be provided to a surgical cavity to a desired dew point, and a flow generator configured to provide the gases flow at a desired flow rate.

### FIELD OF THE DISCLOSURE

The present disclosure relates to a surgical humidification system.

### BACKGROUND

In surgery, insufflation gases can be used for a variety of purposes. In open surgery, gas can be insufflated into a body cavity for de-airing, such as for example in cardiac surgery. In laparoscopic surgery, the surgical cavity wall (for example the abdominal wall) can be distended using gas to provide room for instrument insertion and tissue dissection. It may be desirable to introduce gases into a surgical cavity under controlled operating parameters such as a particular flow rate, pressure, temperature, and/or humidity.

### SUMMARY

In an aspect there is disclosed a surgical humidification system, comprising:
a flow generator configured to control a gases flow to be provided to a surgical cavity at a desired flow rate, wherein the desired flow rate is a flow rate of about 3 litres/minute to about 120 litres/minute,
a humidifier configured to humidify the gases flow to be provided to the surgical cavity, and
wherein the humidifier is configured to humidify the gases flow to a desired dew point of about 35 degrees Celsius to about 43 degrees Celsius, or about 40 degrees Celsius.

In an aspect there is disclosed a surgical humidification system, comprising:
a flow generator configured to control a gases flow to be provided to a surgical cavity at a desired flow rate, wherein the desired flow rate is a flow rate of about 45 litres/minute to about 65 litres/minute,
a humidifier configured to humidify the gases flow to be provided to the surgical cavity, and
wherein the humidifier is configured to humidify the gases flow to a desired dew point of about 38 degrees Celsius to about 41 degrees Celsius.

In some configurations, the humidifier and/or a heater of a patient conduit pneumatically connected to the humidifier, is configured to heat the gases flow to a desired temperature of about 38 degrees Celsius to about 43 degrees Celsius.

In some configurations, the humidifier and/or the heater of a patient conduit is configured to heat the gases flow to a desired temperature, wherein the desired temperature is: about equal to the desired dew point, above the desired dew point, about an offset plus the desired dew point, and/or based on the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In an aspect there is disclosed a surgical humidification system, comprising:
a flow generator configured to control a gases flow to be provided to a surgical cavity at a desired flow rate, wherein the desired flow rate is a flow rate of about 3 litres/minute to about 120 litres/minute,
a humidifier configured to heat and humidify the gases flow to be provided to a surgical cavity,
wherein the humidifier and/or a heater of a patient conduit pneumatically connected to the humidifier, is configured to humidify the gases flow to a desired dew point of about 35 degrees Celsius to about 43 degrees Celsius, or about 40 degrees Celsius, and to heat the gases flow to a desired temperature about equal to the desired dew point, about an offset plus the desired dew point, above the desired dew point, and/or based on the desired dew point.

In an aspect there is disclosed a surgical humidification system, comprising:
a flow generator configured to control a gases flow to be provided to a surgical cavity at a desired flow rate, wherein the desired flow rate is a flow rate of about 45 litres/minute to about 65 litres/minute,
a humidifier configured to heat and humidify the gases flow to be provided to a surgical cavity,
wherein the humidifier and/or a heater of a patient conduit connected to the humidifier is configured to humidify the gases flow to a desired dew point of about 38 degrees Celsius to about 41 degrees Celsius, and to heat the gases flow to a desired temperature of about 38 degrees Celsius to about 43 degrees Celsius, about equal to the desired dew point, about an offset plus the desired dew point, above the desired dew point, and/or based on the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In some configurations, the desired flow rate is provided as a continuous flow rate.

In some configurations, the humidifier has a separate heater and a separate humidification unit, wherein the heater is configured to heat the gases and the humidification unit is configured to humidify the gases.

In some configurations, the dew point is measured at or near an outlet of the humidifier, at or near an end of an outlet of the humidifier, at or near an end of a conduit connected to the humidifier, at or near an inlet of a cannula which is configured to deliver the gases flow to the surgical site, at or near an outlet of a cannula which is configured to deliver the gases flow to the surgical site, or in the surgical cavity.

In some configurations, the gases flow is provided to the surgical cavity at a temperature at or above the desired dew point.

In some configurations, the desired dew point is at or about atmospheric pressure, or at or about atmospheric +the pressure of the surgical cavity, or at or about atmospheric +10 mm Hg.

In some configurations, the system is configured to control a dew point of the gases to the desired dew point.

In some configurations, the dew point of the gases is based on the output of one or more sensors.

In some configurations, the dew point of the gases is estimated based on at least the output of a at least one temperature sensor, and/or at least one flow rate sensor.

In some configurations, the desired dew point is a dew point at a location downstream of the humidifier.

In some configurations, the desired dew point is a dew point at a location:
a) at or near an inlet or outlet of a patient conduit,
b) at or near a patient end or a humidifier end of a patient conduit connected to the humidifier,
c) at or near a beginning of a conduit connected to the humidifier,
d) at or near an inlet of a cannula which is configured to deliver the gases flow to the surgical site,
e) at or near an outlet of a cannula which is configured to deliver the gases flow to the surgical site.
f) In the surgical cavity.

In some configurations, the system is configured to control a temperature of the gases to the desired temperature.

In some configurations, the temperature of the gases is based on the output of one or more sensors.

In some configurations, the desired temperature is at a location:
a) at or near an inlet or outlet of a patient conduit,
b) at or near a patient end or a humidifier end of a patient conduit connected to the humidifier,
c) at or near a beginning of a conduit connected to the humidifier,
d) at or near an inlet of a cannula which is configured to deliver the gases flow to the surgical site,
e) at or near an outlet of a cannula which is configured to deliver the gases flow to the surgical site.
f) In the surgical cavity.

In some configurations, the gases flow is Carbon Dioxide

In some configurations, the gases flow comprises:
a) ambient air,
b) Oxygen,
c) Carbon Dioxide,
d) any combination of a)-c).

In some configurations, the desired dew point and/or a desired gases temperature, and/or the desired flow rate are scaled based on a composition of the gases flow.

In some configurations, the surgical cavity is a pneumoperitoneum of the patient.

In some configurations, the gases flow is diffused as it enters the surgical cavity.

In some configurations, the flow generator and humidifier are provided in a single housing.

In some configurations, the flow generator is an insufflator.

In some configurations, the flow generator comprises a blower.

In some configurations, the flow generator is connected to a pressurised source of gases.

In some configurations, the flow generator comprises a valve configured to provide the gases flow by controlling the outlet of the pressurised source of gases.

In some configurations, the flow generator comprises a controller, and the humidifier comprises a controller.

In some configurations, an outlet of the humidifier is configured to pneumatically connect to a conduit, the conduit is configured to provide a passageway for the gases flow.

In some configurations, the conduit comprises a heater.

In some configurations, the heater of the conduit is configured to maintain the gases temperature at or above the desired dew point.

In some configurations, the heater is a heater wire.

In some configurations, the heater wire is located:
in a passageway of the conduit,
attached to a wall of the conduit
embedded in a wall of the conduit.

In some configurations, the conduit is connected to a cannula, the cannula configured to deliver the gases flow to the surgical cavity.

In some configurations, the system comprises an exhaust line, the exhaust line providing a vent pathway from the surgical cavity.

In some configurations, the exhaust line is connected to an exhaust cannula.

In some configurations, the exhaust line is connected to a suction source.

In some configurations, the system comprises at least one filter, where the filter is located upstream of the flow generator and/or downstream of the humidifier and/or between the flow generator and the humidifier.

In some configurations, the system comprises at least one temperature sensor, the at least one temperature sensor configured to measure a temperature of the gases flow, wherein the at least one temperature sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

In some configurations, the system comprises at least one flow rate sensor, the at least one flow rate sensor configured to measure a flow rate of the gases flow, wherein the at least one flow rate sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

In some configurations, the system comprises at least one pressure sensor, the at least pressure sensor configured to measure a pressure of the gases flow, wherein the at least one pressure sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

In some configurations, the system comprises at least one humidity sensor, the at least one humidity sensor configured to measure a humidity of the gases flow, wherein the at least one humidity sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

In some configurations, the gases flow is recirculated from the surgical cavity through the humidifier.

In some configurations, the gases flow is recirculated from the surgical cavity through the humidifier by the flow generator as a recirculation flow generator.

In some configurations, the recirculation flow generator is configured to provide gases at a or the desired flow rate.

In some configurations, the gases flow is recirculated through a recirculation circuit, the recirculation circuit comprising a humidifier and the flow generator.

In some configurations, the system comprises a primary flow generator (optionally an insufflator) configured to control the volume and/or pressure of the surgical cavity of the patient, and wherein the flow generator is a secondary flow generator.

In some configurations, the primary flow generator (optionally the insufflator) is configured to provide a primary flow of gases to the surgical cavity of the patient.

In some configurations, the primary flow generator (optionally the insufflator) is connected to an insufflation cannula, wherein the insufflation cannula provides the primary flow of gases to the surgical cavity of the patient.

In some configurations, the system comprises a primary humidifier configured to humidify the primary flow of gases, and wherein the humidifier is a secondary humidifier.

In some configurations, the primary flow generator (optionally the insufflator) and primary humidifier are provided in a single housing.

In some configurations, the system comprises a gases recirculation circuit, the gases recirculation circuit comprising a recirculation flow generator as the flow generator, configured to recirculate gases from the surgical cavity of the patient through a humidifier and back to the surgical cavity of the patient.

In another aspect there is disclosed a surgical humidification system, comprising: a gases recirculation circuit, the gases recirculation circuit comprising a recirculation flow generator configured to recirculate gases from a surgical cavity of the patient through a humidifier and back to the surgical cavity of the patient.

In some configurations, the recirculation flow generator is configured to recirculate the gases through the gases recirculation circuit.

In some configurations, the humidifier is configured to humidify the recirculated gases in the recirculation circuit.

In some configurations, the recirculation flow generator is configured to control the volume and/or pressure of the surgical cavity of the patient.

In some configurations, the system comprises the primary flow generator, the primary flow generator is configured to control the volume and/or pressure of the surgical cavity of the patient and wherein the recirculation flow generator is a secondary flow generator.

In some configurations, the primary flow generator is an insufflator to control a separate primary gases flow to be provided to the surgical cavity of a patient.

In some configurations, the system comprises at least one recirculation cannula.

In some configurations, the at least one recirculation cannula comprises a single recirculation cannula, the single recirculation cannula comprising an outlet gas pathway providing a pathway for gases from the surgical cavity and an inlet gas pathway providing a pathway for gases to the surgical cavity.

In some configurations, at least one recirculation cannula comprises an inlet cannula providing an inlet gases pathway for gases to the surgical cavity and an outlet cannula providing an outlet gases pathway for gases from the surgical cavity.

In some configurations, the system comprises a recirculation supply conduit configured to pneumatically connect the inlet gases pathway of the at least one recirculation cannula to an outlet of the humidifier.

In some configurations, the system comprises a recirculation return conduit configured to pneumatically connect the outlet gases pathway of the at least one recirculation cannula to an inlet of the recirculation flow generator.

In some configurations, the recirculation supply conduit and/or the recirculation return conduit comprise a heater.

In some configurations, the heater is a heater wire.

In some configurations, the heater wire is located:
in a passageway of the conduit,
attached to a wall of the conduit,
embedded in a wall of the conduit.

In some configurations, the system is configured to control at least one gases parameter of the gases flow to at least one first desired gases parameter if a patient heating criterion is not satisfied, and
wherein the system is operable to control the at least one gases parameter of the gases flow to at least one second desired gases parameter if a patient heating criterion is satisfied.

In another aspect there is disclosed a surgical humidification system, comprising:
a flow generator configured to control a gases flow to be provided to a surgical cavity of a patient,
a humidifier, wherein the humidifier heats and/or humidifies the gases flow,
wherein the system is configured to control at least one gases parameter of the gases flow to at least one first desired gases parameter so as to warm the patient, and
wherein the system is operable to control the at least one gases parameter of the gases flow to at least one second desired gases parameter when the patient does not require heating.

In some configurations, the system is configured to operate in a smoke evacuation mode, wherein in the smoke evacuation mode the system is configured to:
maintain a humidity level and/or a temperature of the gases
increase the flow rate of the gases so as to remove smoke from the surgical cavity.

In some configurations, the system is configured to:
control the system according to one or more modes, each mode defining one or more operating parameters, and/or operating parameter limits, identify a cannula connected to the system, and
control the system according to a mode associated with the identified cannula of the one or more modes.

In some configurations, the operating parameters, and/or operating parameter limit of the mode associated with the identified cannula is/are based on, at least in part, characteristics of the identified cannula.

In some configurations, the characteristics are one or more physical characteristics and/or one or more operational characteristics

In some configurations, the one or more physical characteristics comprise the presence of diffusion apertures in the cannula.

In some configurations, the one or more physical characteristics comprise a maximum allowed flow rate, dew point and/or gases temperature for the cannula.

In another aspect there is disclosed a surgical humidification system, comprising:
a flow generator configured to control a gases flow to be provided to a surgical cavity of a patient,
a humidifier, wherein the humidifier heats and/or humidifies the gases flow,
wherein the system is configured to control at least one gases parameter of the gases flow to lower heat transfer to the patient when patient heating is not required and to control the at least one gases parameter of the gases flow to increase heat transfer to the patient when patient heating is required.

In another aspect there is disclosed a surgical humidification system, comprising:
a flow generator configured to control a gases flow to be provided to a surgical cavity of a patient,
a humidifier, wherein the humidifier heats and/or humidifies the gases flow,
wherein the system is configured to control at least one gases parameter of the gases flow to at least one first desired gases parameter if a patient heating criterion is not satisfied, and
wherein the system is operable to control the at least one gases parameter of the gases flow to at least one second desired gases parameter if a patient heating criterion is satisfied.

In some configurations, one or more of the at least one second desired gases parameter is lower than the corresponding at least one first desired gases parameter.

In some configurations, the at least one gases parameter comprises any one or combination of: a dew point of the gases flow, a temperature of the gases flow, or a flow rate of the gases flow.

In some configurations, the at least one first desired gases parameter comprises a first desired dew point and/or a first desired temperature and/or a first desired flow rate.

In some configurations, the at least one second desired gases parameter comprises a second desired dew point and/or a second desired temperature and/or a second desired flow rate.

In some configurations, the second desired dew point is lower than the first desired dew point.

In some configurations, the second desired flow rate is lower than the first desired flow rate.

In some configurations, the second desired temperature is lower than the first desired temperature.

In some configurations, the first desired flow rate is about 40 litres/minute to about 55 litres/minute, or about 65 litres/minute.

In some configurations, the first desired dew point is about 35 degrees Celsius to about 43 degrees Celsius, or about 38 degrees Celsius to about 41 degrees Celsius, or about 40 degrees Celsius.

In some configurations, the first desired temperature is about equal to the first desired dew point, above the first desired dew point, about an offset plus the first desired dew point, and/or based on the first desired dew point.

In some configurations, the first desired temperature is 38 degrees Celsius to about 43 degrees Celsius.

In some configurations, the second desired flow rate is about 10 litres/minute to about 30 litres/minute, or about 10 litres/minute.

In some configurations, the second desired flow rate is above a predetermined threshold.

In some configurations, the predetermined threshold is 6 litres/minute, or about 10 litres/minute.

In some configurations, the second desired dew point is about 33 degrees Celsius to about 37 degrees Celsius, or about 34 degrees Celsius, or about 37 degrees Celsius.

In some configurations, the second desired temperature is about equal to the second desired dew point, above the second desired dew point, about an offset plus the second desired dew point, and/or based on the second desired dew point.

In some configurations, the second desired temperature is 37 degrees Celsius to about 43 degrees Celsius, or above the desired dew point.

In some configurations, the system is configured to determine that a patient does, or does not require heating is based on an input from a user.

In some configurations, the input is from a user interface.

In some configurations, the system is configured to determine that a patient does, or does not need heating based on the output of a patient temperature sensor.

In some configurations, the patient temperature sensor is configured to measure a patient's core body temperature, or an external patient temperature.

In some configurations, the system is configured to determine that a patient does not need heating when the patient's core body temperature and/or the external patient temperature is above a threshold.

In some configurations, the system is configured to determine that a patient does need heating when the patient's core body temperature and/or the external patient temperature is below a threshold.

In some configurations, the system comprises a gases recirculation circuit, the gases recirculation circuit comprising the flow generator as a recirculation flow generator configured to recirculate gases from the surgical cavity of the patient through the humidifier and back to the surgical cavity of the patient.

In some configurations, the system comprises a venting cannula.

In some configurations, the system comprises a heater of a patient conduit pneumatically connected to the humidifier configured to heat and/or humidify the gases flow in addition to the humidifier.

In another aspect there is disclosed a method of transferring heat to a patient undergoing a surgical procedure to protect a patient from unintended perioperative hypothermia, the method comprising:
conditioning a gases flow, wherein conditioning the gases flow comprises humidifying the gases flow to a dew point higher than the patient's core body temperature and heating the gases flow to a temperature at or above the dew point, and
delivering the gases flow to a surgical cavity of a patient.

In some configurations, conditioning the gases flow further comprises controlling the gases flow to a flow rate of about 3 litres/minute to about 120 litres/minute, or about 45 litres/minute to about 65 litres/minute.

In some configurations, the dew point the gases are humidified to is about 35 degrees Celsius to about 43 degrees Celsius, or about 38 degrees Celsius to about 41 degrees

In some configurations, the gases flow is heated to a desired temperature, wherein the desired temperature is: about equal to a desired dew point, above a/the desired dew point, about an offset plus a/the desired dew point, and/or based on a/the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In some configurations, the gases flow is provided from a flow generator.

In another aspect there is disclosed a method of transferring heat to a patient to protect a patient from unintended surgery-related hypothermia, the method comprising:
conditioning a gases flow, wherein conditioning the gases flow comprises humidifying the gases flow to a dew point higher than the patient's core body temperature and heating the gases flow to a temperature at or above the dew point, and
delivering the gases flow to a conduit.

In some configurations, the conduit is suitable for inclusion in a cavity of a patient.

In some configurations, the gases flow is delivered to the conduit at a flow rate of about 3 litres/minute to about 120 litres/minute, or about 45 litres/minute to about 65 litres/minute.

In some configurations, the dew point the gases are humidified to is about 35 degrees Celsius to about 43 degrees Celsius, or about 38 degrees Celsius to about 41 degrees

In some configurations, the gases flow is heated to a desired temperature, wherein the desired temperature is: about equal to a desired dew point, above a/the desired dew point, about an offset plus a/the desired dew point, and/or based on a/the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In another aspect there is disclosed a method of conditioning a gas flow, the method comprising:
humidifying the gases flow to a dew point wherein the dew point is about 35 degrees Celsius to about 41 degrees Celsius, or about 38 degrees Celsius to about 41 degrees Celsius, and heating the gases flow to a temperature at or above the dew point.

In some configurations, the gases flow is at a flow rate of about 3 litres/minute to about 120 litres/minute, or about 45 litres/minute to about 65 litres/minute.

In some configurations, the dew point the gases are humidified to is about 35 degrees Celsius to about 43 degrees Celsius, or about 38 degrees Celsius to about 41 degrees

In some configurations, the gases flow is heated to a desired temperature, wherein the desired temperature is: about equal to a desired dew point, above a/the desired dew point, about an offset plus a/the desired dew point, and/or based on a/the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In some configurations, the method is provided by the system of any of the above aspects.

In another aspect there is disclosed a composition comprising carbon dioxide having a dew point of about 35 degrees Celsius to about 41 degrees Celsius, or about 38 degrees Celsius to about 43 degrees Celsius and at a temperature of about 38 degrees Celsius to about 41 degrees, an offset plus the dew point, or above the dew point, for use in a method of transferring heat to a patient undergoing a surgical procedure to protect a patient from unintended perioperative hypothermia, the method comprising delivering the composition to the surgical cavity of a patient.

In some configurations, the composition is delivered at a flow rate of 3 litres/minute to about 120 litres/minute, or about 45 litres/minute to about 65 litres/minute.

In some configurations, in addition to carbon dioxide the composition comprises ambient air and/or Oxygen.

In another aspect there is disclosed a surgical humidification system, comprising:
a flow generator configured to control a gases flow to be provided to a surgical cavity,
a humidifier configured to humidify the gases flow to be provided to the surgical cavity,
wherein the gases flow is provided at a flow rate and humidity such that condensation of water vapour in the surgical cavity heats the patient during a surgical procedure.

In some configurations, the flow rate is about 3 litres/minute to about 120 litres/minute, or about 45 litres/minute to about 65 litres/minute.

In some configurations, the humidity is a dew point, and the gases are humidified to is about 35 degrees Celsius to about 43 degrees Celsius, or about 38 degrees Celsius to about 41 degrees.

In some configurations, the gases flow is heated to a desired temperature, wherein the desired temperature is: about equal to a desired dew point, above a/the desired dew point, about an offset plus a/the desired dew point, and/or based on a/the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In another aspect there is disclosed a surgical humidification system, comprising:
a flow generator configured to control a gases flow to be provided to a surgical cavity to a desired flow rate,
a humidifier, wherein the humidifier heats and humidifies the gases to a desired dew point,
wherein the system is configured to transfer energy to the patient, wherein the energy is provided by at least condensation of water vapour on tissue of the patient within the surgical cavity, wherein the energy transferred to the patient is based on at least: the desired flow rate and the desired dew point.

In some configurations, the desired flow rate is about 3 litres/minute to about 120 litres/minute, or about 45 litres/minute to about 65 litres/minute.

In some configurations, the desired dew point is about 35 degrees Celsius to about 43 degrees Celsius, or about 38 degrees Celsius to about 41 degrees.

In some configurations, the gases flow is heated to a desired temperature, wherein the desired temperature is: about equal to a desired dew point, above a/the desired dew point, about an offset plus a/the desired dew point, and/or based on a/the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In another aspect there is disclosed a surgical humidification system, comprising:
a flow generator configured to control a gases flow to be provided to a surgical cavity,
a humidifier configured to humidify the gases flow to be provided to the surgical cavity,
a gases recirculation circuit, the gases recirculation circuit comprising a recirculation flow generator configured to recirculate gases from the surgical cavity of the patient through a humidifier and back to the surgical cavity of the patient,
wherein the gases flow is provided at a flow rate, humidity and/or temperature so as to reach or maintain normothermia for a patient.

In some configurations, the desired flow rate is about 3 litres/minute to about 120 litres/minute, or about 45 litres/minute to about 65 litres/minute.

In some configurations, the desired dew point is about 35 degrees Celsius to about degrees Celsius, or about 38 degrees Celsius to about 41 degrees

In some configurations, the gases flow is heated to a desired temperature, wherein the desired temperature is: about equal to a desired dew point, above a/the desired dew point, about an offset plus a/the desired dew point, and/or based on a/the desired dew point.

In some configurations, the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

In another aspect there is disclosed a surgical humidification system, comprising:
a flow generator configured to control a gases flow to be provided to a surgical cavity to a desired flow rate,
a humidifier, wherein the humidifier heats and humidifies the gases to a desired dew point,
wherein the system is configured to transfer energy to the patient, wherein the energy is provided by at least condensation of water vapour on tissue of the patient within the surgical cavity, wherein the energy transferred to the patient is based on at least: the desired flow rate and the desired dew point.

In another aspect there is disclosed a surgical humidification system, comprising:
a flow generator configured to control a gases flow to be provided to a surgical cavity to a desired flow rate,
a humidifier, wherein the humidifier heats and humidifies the gases to a desired dew point,
wherein the system is configured to operate in a smoke evacuation mode, wherein in the smoke evacuation mode the system is configured to:
maintain a humidity level and/or a temperature of the gases
increase the flow rate of the gases so as to remove smoke from the surgical cavity.

In another aspect there is disclosed a surgical humidification system, comprising:
a flow generator configured to control a gases flow to be provided to a surgical cavity to a desired flow rate,
a humidifier, wherein the humidifier heats and humidifies the gases to a desired dew point,
wherein the system is configured to:
control the system according to one or more modes, each mode defining one or more operating parameters, and/or operating parameter limits, identify a cannula connected to the system, and
control the system according to a mode associated with the identified cannula of the one or more modes.

In some configurations, the operating parameters, and/or operating parameter limit of the mode associated with the identified cannula is/are based on, at least in part, characteristics of the identified cannula.

In some configurations, the characteristics are one or more physical characteristics and/or one or more operational characteristics.

In some configurations, the one or more physical characteristics comprise the presence of diffusion apertures in the cannula.

In some configurations, the one or more physical characteristics comprise a maximum allowed flow rate, dew point and/or gases temperature for the cannula.

In another aspect there is disclosed a method of controlling a surgical humidification system, the method comprising:
controlling the system to humidify a gases flow to a dew point of about 38 degrees Celsius to about 41 degrees Celsius, and
controlling the system so that the flow rate of the gases flow is about 3 litres/minute to about 120 litres/minute.

In another aspect there is disclosed a method of controlling a surgical humidification system, the method comprising:
controlling the system to humidify a gases flow to a dew point of about 38 degrees Celsius to about 41 degrees Celsius, and
controlling the system so that the flow rate of the gases flow is about 3 litres/minute to about 120 litres/minute
providing the gases flow to a conduit.

In some configurations, the method comprises delivering the gases flow to a patient interface and/or conduit.

In some configurations, the method comprises delivering the gases flow to a patient cavity via the conduit and/or patient interface.

In some configurations, the method comprises controlling the system to heat the gases flow to a temperature about equal to the dew point, about an offset plus the dew point, above the dew point, and/or based on the dew point.

In another aspect there is disclosed a method of providing a flow of gases to a surgical cavity, the method comprising:
conditioning the flow of gases according to one or more operational parameters, wherein the one or more operational parameters comprise one or more of:
a desired dew point of the flow of gases,
a desired gases temperature of the flow of gases, and/or
a desired flow rate of the flow of gases, and
monitoring one or more patient parameters and/or environmental parameters,
increasing heating, by increasing one or more operational parameters,
maintaining heating, by maintaining one or more operational parameters,
decreasing heating, by decreasing one or more operational parameters.

In another aspect there is disclosed a method of providing a flow of gases to a surgical cavity, the method comprising:
conditioning the flow of gases according to one or more operational parameters, wherein the one or more operational parameters comprise at least the dew point of the flow of gases,
monitoring a patient temperature, and
increasing the dew point of the flow of gases if the patient temperature is below a threshold, and/or
maintaining the dew point of the flow of gases if the patient temperature is within an acceptable range,
decreasing the dew point of the flow of gases if the patient temperature is above a threshold.

In another aspect there is disclosed a method of providing a flow of gases to a surgical cavity, the method comprising:
conditioning the flow of gases according to one or more operational parameters, wherein the one or more operational parameters comprise: a desired dew point of the flow of gases, a desired gases temperature of the flow of gases, and/or a desired flow rate of the flow of gases, and
monitoring a patient temperature, and
increasing one or more operational parameters of the flow of gases if the patient temperature is below a threshold,
maintaining one or more operational parameters of the flow of gases if the patient temperature is within an acceptable range, and/or
decreasing one or more operational parameters of the flow of gases if the patient temperature is above a threshold.

In another aspect there is disclosed a method of providing a flow of gases to a surgical cavity, the method comprising:
monitoring a patient temperature,
conditioning the flow of gases according to one or more operational parameters, wherein the one or more operational parameters comprise at least the dew point of the flow of gases, wherein the dew point of the flow of gases is above the monitored patient temperature, and
delivering the gases flow to a patient cavity.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 illustrates a schematic view of an example of a system for providing humidified gases to a surgical cavity.

FIG. 2 illustrates a schematic view of an example of a system for providing humidified gases to a surgical cavity which includes a recirculation circuit and a flow generator.

FIG. 3 illustrates a system for providing humidified gases to a surgical cavity which includes a recirculation circuit and a flow generator.

FIGS. 4 and 5 illustrate schematic views of an example of a system for providing humidified gases to a surgical cavity which includes a recirculation circuit.

FIG. 6 illustrates a schematic view of an example of a system for providing humidified gases to a surgical cavity which comprises two flow generators.

FIG. 11 illustrates a cross section of an example of a surgical cavity.

FIG. 12 illustrates a view of a cannula.

FIG. 12A illustrates an example of an end of a cannula.

FIGS. 13 and 14 illustrates a flow diagram for conditioning gases based on whether a patient requires heating.

FIG. 15 illustrates a flow diagram of an example for determining whether a patient requires heating.

FIG. 16 shows a flow chart outlining an example of control of the system.

FIG. 17 shows an example graph of patient temperature over time.

FIG. 18 shows an example of the system operating in a particular mode based on whether or not a compatible cannula is detected.

FIG. 19 shows an example of operation of the smoke evacuation mode.

FIGS. 20 and 21 illustrate a flow diagram for a method of heating a patient.

### DETAILED DESCRIPTION

In the following description, numerous specific details are set forth in order to provide a thorough understanding of the various principles of the disclosure. However, those skilled in the art will appreciate that not all these details are necessarily always required for practicing embodiments according to the disclosure.

During surgical procedures (such as laparoscopic and endoscopic procedures) patients may be subject to various factors which may lead to heat loss. Heat loss may lower the core body temperature of the patient which may have an adverse effect on the patient (for example myocardial injury, surgical site infection, blood loss and transfusion, length of hospital stay and re-admission).

Heat loss from a patient may occur for a number of reasons such as for example: cold operating theatres, drafts of air in the operating theatre (for example caused by surgical equipment to create a sterile environment), using a cold gas in insufflation, lack of insulation of the patient (for example skin exposed to the ambient environment), the administration of anesthetic drugs, and/or the provision of intravenous fluids which may be of a temperature lower than the patient temperature.

The disclosure provides a system that transfers heat to the patient to help increase, regulate and/or maintain the patient's core body temperature.

The gases may be provided to the surgical cavity such that when the gas enters the surgical cavity water vapour energy is transferred to the patient.

Energy may be transferred to the patient through multiple heat transfer mechanisms. One mechanism may be condensation of water vapour from the gases onto patient tissue. Condensation of the water vapour transfers heat to the patient.

Another mechanism may be heat transfer between the gases and the patient tissue.

Characteristics of the gas which may be controlled to determine the overall energy transferred to the patient may include one or more of: dew point temperature, gases temperature, and/or flow rate of the gases.

Factors relating to how the gases are delivered to the surgical cavity, for example diffusion of the gases via a diffuser (described in more detail below), flow velocity or flow turbulence, may also have an effect on the overall energy transferred to the patient.

Providing humidified gases at a dew point greater than a patient's core body temperature, (for example 1 to 10 degrees Celsius above a patient core body temperature, or about 1 to 5 degrees Celsius above a patient core body temperature), or above about 35 degrees Celsius (35° C. and below may be considered to be clinically hypothermic), allows for the transfer of energy from the gases to the patient through condensation of the water vapour in the gases in the surgical cavity of the patient. The gases can be provided to the surgical cavity at a dew point greater than, for example, about 35 degrees Celsius and as they enter the surgical cavity the gases are cooled (for example by the surrounding tissue of the patient) to a temperature at or below the dew point and the water vapour of the gases condenses out from the gas. The condensation of the water vapour transfers heat to the patient. The humidified gases may be provided at a dew point as described elsewhere in the specification.

Further heat transfer may occur between the gases and the patient by for example convection and/or conduction.

The temperature of the gases provided to the surgical cavity may impact the amount of heat transferred from the gases to the patient. For example, the higher the temperature of the gases, the higher the temperature gradient between the gases and the patient, and the higher the heat transfer. However, it will be appreciated that increasing the gases temperature too much may lead to a risk to the patient, and it has been found that increasing the temperature alone cannot introduce sufficient energy to the patient to regulate and/or maintain the patient's core body temperature. A further consideration is that depending on the humidity content of the gas, increasing the gases temperature may increase evaporation, which may remove heat and/or moisture from the patient.

The flow rate of the gases provided to the surgical cavity may impact the amount of heat transferred from the gases to the patient. For example, the higher the flow rate the more frequently gases are replaced in the surgical cavity. Generally, this means that the higher the flow rate the higher the energy transferred to the patient (for example, the increased volume of gases, and amount of water vapour provided to the surgical cavity can increase the chance for water vapour to condense on a surface of the patient cavity, such as tissue surface). However, it has been found that increasing the flow rate too much may lead to diminishing returns in terms of heat transfer (by decreasing dwell time in the surgical cavity and therefore the time where heat can be transferred from the gases, and for example, by decreasing the chance for water vapour to condense onto a surface of the patient cavity.).

Flow through the surgical cavity may also act to help remove smoke from the cavity generated by a surgical procedure. For example, providing a higher flow rate, and optionally a recirculating flow, can help to increase the rate at which gases in the surgical cavity are removed and filtered, so as to help with removing smoke from the surgical cavity.

Current insufflation systems provide flow rates which are typically lower than those described in the present disclosure. Further, humidification of the gases provided to the surgical cavity is not always provided, and often cold dry gases are provided to the surgical cavity which may increase the risk of detrimental effects to the patient (for example drying out of patient tissue). Higher flow rates, such as those described in the disclosure, provided in systems without a humidifier may increase the risk of detrimental effects to the patient, and therefore, in systems without a humidifier, flow rates of gases provided to the surgical cavity may be kept as low as possible. Low flow insufflation during surgery may be considered to be associated with fewer changes in cardiovascular function, better patient satisfaction scores and lower postoperative pain.

The flow rates utilised in the system disclosed in the present disclosure are generally higher than currently available surgical gases delivery systems. For example, surgical insufflation typically provides cold and dry gases, at lower flows than as described in the present disclosure.

Some systems do have a humidifier to humidify the flow of gases before they are provided to the patient. Current surgical humidification systems humidify gases which are provided to the surgical cavity by an insufflator. The insufflator's purpose is to establish and maintain the surgical cavity volume and pressure to increase operative space and visualization for surgeons, and to do so does not need to provide higher gas flows. The current disclosure discloses systems which include an additional flow generator in addition to the insufflator, systems which operate independently of an insufflator, and systems without an insufflator.

Further, current surgical humidification systems do not provide gases to the surgical cavity at a combination of flow rate and dew point which would provide for energy transfer to the patient to increase/regulate/maintain core body temperature.

Further, the current disclosure may provide for a system that more consistently warms the patient, relative to external warming of the patient, as external warming may be dependent on how well the device providing the warming is applied to the patient.

The current disclosure may also provide for increased smoke evacuation due to the relatively high flow rates provided to the surgical cavity which may help to clear any smoke generated from the surgical procedure from the surgical cavity. This may help to maintain optical clarity in the surgical cavity for the clinician.

The disclosure may provide a system, method and/or apparatus that provides an overall positive, or neutral heat flux gradient to the patient. That is the patient is being provided with more energy than they are losing. The disclosure may also at least reduce the heat loss of the patient. This may not be possible with prior art systems, which provide energy but do not provide enough to offset losses (for example environmental losses).

The disclosure may help to protect a patient from unintended surgery-related hypothermia.

The disclosure also provides a system comprising a recirculation circuit which recirculates gases from the surgical cavity. Gases recirculated from the surgical cavity may have already been previously heated and humidified, including by the patient, and so less energy needs to be expended by the humidifier to heat and humidify the gases to the desired temperature and dew point. Recirculation of gases from the surgical cavity may therefore increase efficiency of the system as the humidifier does not have to heat and humidify gases from source conditions which may be low temperature and/or low humidity—i.e. cold and dry (for example surgical gases provided from a gases source).

Recirculation of gases from the surgical cavity may also decrease the cost of the gases (for example Carbon Dioxide) as it reduces the amount of gases vented to atmosphere. If gases can be recirculated rather than being vented to atmosphere, then less fresh gas is required to be added to the system. This may be of particular importance at higher flow rates, as a constant supply of fresh gases would be needed.

The system of the disclosure may be used with other techniques to try and limit heat loss from a patient before, during and/or after surgery. For example, heating systems may be used to transfer heat to an external part of the patient's body before, during, or after surgery. Heat may be transferred to an external part of the patient's body by for example forced air warming, conductive heating. In some examples the heating system may transfer heat via warmed IV fluids which are provided to the patient for example via an Intravenous cannula. The system of the disclosure may for example be used to transfer heat to a patient during surgery, and other heating systems may be used to transfer heat to a patient before, during and/or after surgery. In some examples, the system of the disclosure may be used in conjunction with other heating systems during surgery.

In the system of the disclosure, cannulas are used to deliver gases to the surgical cavity. Different cannulas may be able to provide therapy to the patient with different operating parameters, for example due to differences in features and/or design. The disclosure describes different modes that the system can operate in depending on the type of cannula used. One or more operating parameters of the system may be changed, depending on the cannula identified.

Evacuation of smoke from the surgical cavity may be inherent to operation of the system of the disclosure (for example as a result of the flow of gases through the surgical cavity). However, also disclosed is a smoke evacuation mode which may increase the flow rate of the gases of the system and optionally change other operating parameters of the system so as to further reduce smoke in the surgical cavity or maintain smoke clearance during therapy.

As discussed above, it will be appreciated that when the system is providing therapy, smoke may be reduced or cleared due to one or more of the: the flow rates provided (which are higher than in current surgical gases delivery systems), and/or in some examples use of the recirculation circuit.

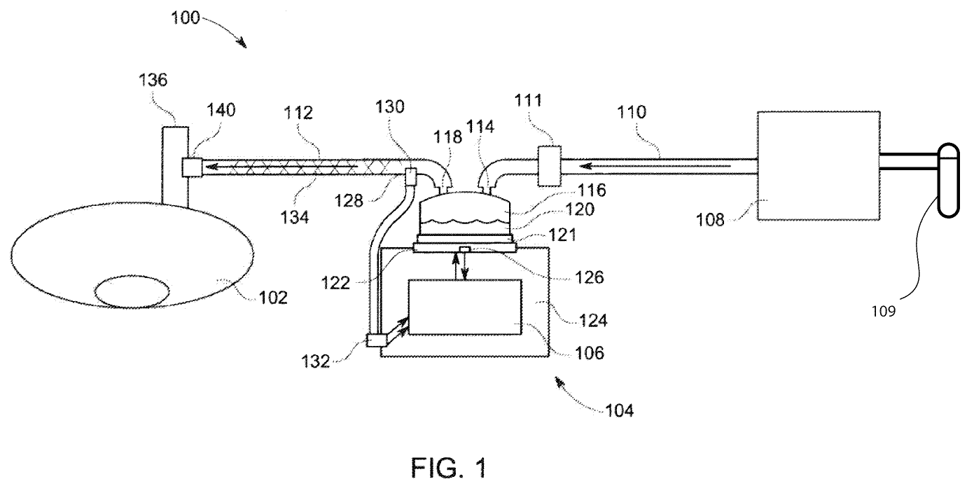

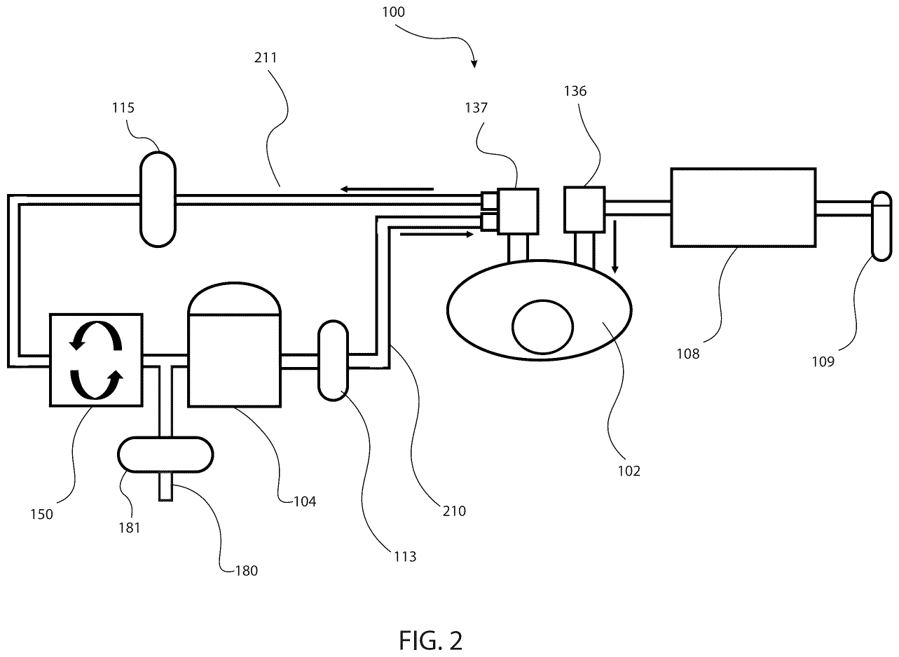

FIG. 1 illustrates an example surgical humidification system 100. The surgical humidification system 100 is an example of an insufflation type surgical gases delivery system. The surgical humidification system 100 delivers gases flow to a surgical cavity of a patient 102. As an example, the surgical cavity may be formed by the provision of the gases flow to the peritoneal cavity of a patient to form a pneumoperitoneum, however it will be appreciated that the surgical cavity may be formed at any location in a patient's body (for example as part of a closed or minimally invasive surgery). The gases are conditioned before being provided to the surgical cavity. Parameters (for example operational parameters) of the gases flow may be controlled by the system (optionally taking into account input from a user for example a clinician), for example any one or combination of gases temperature, gases humidity (i.e. gases dew point), gases flow rate, and/or gases pressure. It will be appreciated that description of the system 100 of FIG. 1 (and for example components of the system for example the humidifier 104 and/or flow generator 108, may apply to other disclosed systems (for example those shown in FIGS. 2-10.)

The system may comprise one or more controllers, for example a system controller, a flow generator controller and/or a humidifier controller. In some configurations, the controller may be associated with any component of the system. The system may be controlled by any one or combination of controllers.

In some configurations, the system controller may be located in a separate housing from other components. In some configurations, the flow generator controller may be located in the flow generator. In some configurations, the humidifier controller may be located in the humidifier. In some configurations, a gases source controller may be located in the gases source.

The controllers may control one or more components of the system. The controllers may control one or more of: the humidifier, the one or more heaters of the system (for example a conduit heating wire and/or a heating element of a filter) and/or the flow generator(s).

The system controls the humidity of the gases to a desired dew point. However, it will be appreciated that other measures of humidity may be controlled, for example, absolute humidity, or relative humidity. The system also controls the flow rate of the gases to a desired flow rate. In some examples the system controls the temperature of the gases to a desired temperature.

The gases flow may be carbon dioxide (e.g. medical grade carbon dioxide). In some examples the gases flow may comprise any one or combination of: ambient air, Oxygen, Carbon Dioxide, one or more inert gases (e.g. argon, helium and nitrous oxide).

In some examples, the desired dew point and/or a desired gases temperature, and/or the desired flow rate are scaled based on a composition of the gases flow.

The surgical humidification system 100 comprises a humidifier 104. The humidifier 104 may humidify and heat the gases flow to the patient 102.

The humidifier may be a passover humidifier which comprises a chamber of water which is heated to humidify gases flowing through the chamber (as described below). In some examples the humidifier may be a humidifier with a wicking or absorbent material which retains humidification fluid, and is heated to humidify the gases. In other examples, the humidifier may be an ultrasonic humidifier, or vapouriser. In other examples, the humidifier may include a nebuliser (for example vibrating mesh or ultrasonic), and/or comprise a mechanism that disturbs liquid in a liquid reservoir to cause at least a portion of the liquid to be in a vapour or aerosolised state to produce humidity.

In some configurations, the humidifier may be an in-line humidifier as part of, or connected with the conduits of the system. The in-line humidifier may comprise an inlet connector and outlet connector which are configured to connect with conduits of the system.

In some configurations, the humidifier may comprise a reservoir which contains a liquid. The reservoir may have an inlet for receiving a liquid (for example a liquid feed bag.) In some configurations, the liquid is water.

The humidifier may also be a component located in a cannula and/or conduit of the system and not a standalone device as described below.

The humidifier 104 is connected to a flow generator 108 through an inlet conduit 110. The humidifier 104 humidifies the gas delivered to the patient 102, and the gases are provided to the patient through a patient or gas delivery conduit 112. The conduits 110, 112 can be made of, for example, flexible plastic tubing. The conduits 110, 112 comprise part of a gases pathway being a gas flow path for delivering gas from the flow generator 108 to the patient (via the humidifier 104).

The patient conduit 112 may be comprised of one or more conduits or tubes.

The inlet conduit 110 may be comprised of one or more conduits or tubes.

The flow generator may be controlled to provide the flow of gases at a desired flow rate and/or a desired pressure. The flow generator may be controlled by a separate flow generator controller, by a system controller, by a humidifier controller 106, or by any combination of controllers.

In some examples, the flow generator may control the gases to both a desired flow rate and a desired pressure.

The flow generator may control the flow of gases to the desired flow rate and/or the desired pressure based on flow rate and/or pressure sensors.

The flow generator may be configured to receive gases from one or more sources. In some examples, the flow generator receives gases from a gas source 109 for example (for example a hospital compressed gas source).

In some examples, the flow generator may receive gases from the ambient environment, which may be filtered.

The flow generator 108 could be integral to the system 100. In some examples, the flow generator comprises a valving arrangement and is configured to generate the flow of gases by a valving arrangement (for example a pressure and/or flow regulating valve).

In some examples, the flow generator comprises a blower and is configured to generate the flow of gases by the blower. The blower may be controlled to generate the flow of gases at the desired flow rate or the desired pressure.

In some cases, both a valving arrangement and a blower may be used.

The gases source 109 may be pressurised. If the gases source is pressurised, no blower may be needed to generate the gases flow, and instead valving arrangements can be used to control the flow of gases by for example controlling valves to generate a desired flow or desired pressure.

In some examples, the flow generator 108 and the humidifier 104 may be separate apparatuses, or be combined to form a single apparatus or the two separate components may be modular and configured to couple together without the need for separate conduit(s) therebetween, as is known in the art.

In some examples, the flow generator 108 may be, or have at least some of the functions of an insufflator.

The flow generator 108 may be configured to control one or more characteristics of the surgical cavity of the patient. The flow generator 108 may be configured to control a volume and/or a pressure of the surgical cavity (for example a pneumoperitoneum).

In some examples, as described in more detail below, the flow generator may be configured to receive gases from the surgical cavity so as to recirculate the gases to the surgical cavity. In these examples, the flow generator may be a recirculation flow generator as described in more detail below. The recirculation flow generator may have any of the features as described elsewhere in the specification with respect to the flow generator 108.

In some examples, and as described in more detail below, the system 100 may comprise multiple flow generators. In this context the system may have a primary flow generator and a secondary flow generator. The primary flow generator may control a primary gases flow to be provided to the surgical cavity, and the secondary flow generator may control a secondary gases flow to be provided to the surgical cavity. The terms primary and secondary in this context do not necessarily denote importance. In some examples, the primary flow generator may control a volume and/or a pressure of the surgical cavity and the secondary flow generator may be a recirculation flow generator.

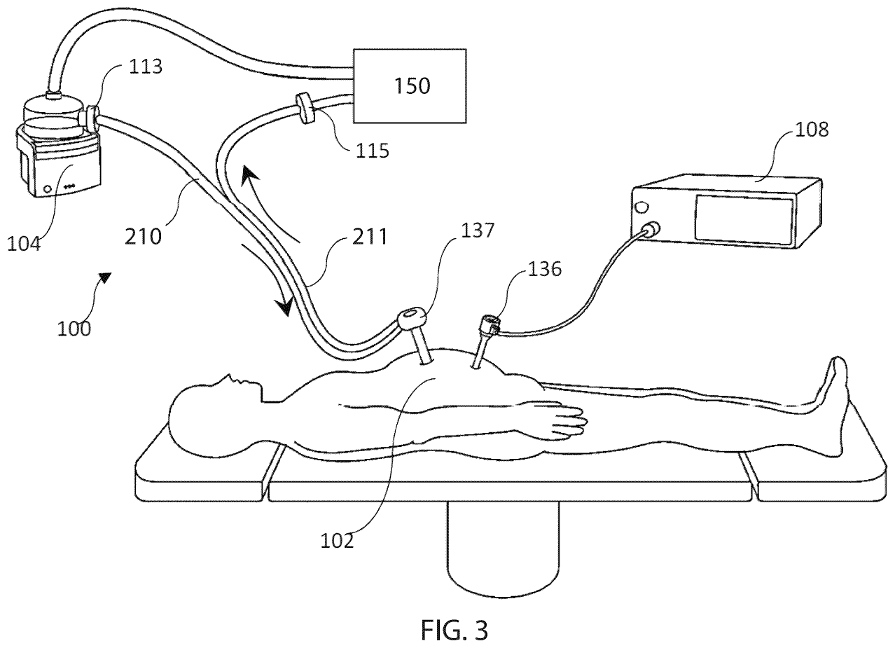

The system 100 may be integrated with another surgical gases delivery system to operate alongside each other. For example, the system 100 may be integrated with an insufflator (for example as the primary flow generator) as shown for example in FIG. 3. The system 100 may operate separately to the insufflator (for example having different controllers which may or may not communicate with each other). The insufflator may have a separate gases pathway via a conduit and cannula to the surgical cavity and controls the volume and/or a pressure of the surgical cavity while the surgical humidification part of the system (for example the recirculation flow generator and associated circuit) operates independently.

In some examples, and as described in more detail below, the system 100 may comprise a single flow generator. In this context the flow generator may control a volume and/or a pressure of the surgical cavity and be a recirculation flow generator.

In some examples, such as the system 100 for example as shown in FIG. 2 and FIG. 3, the system 100 may comprise a primary flow generator 108 and a recirculation flow generator (which may be also referred to as a secondary flow generator in systems with a multiple flow generator). The primary flow generator 108 may be configured to control a volume and/or a pressure of the surgical cavity (for example a pneumoperitoneum), while the recirculation flow generator 150 may be configured to recirculate gases from the surgical cavity back to the surgical cavity.

Referring again to FIG. 1, the humidifier 104 can receive gas from the flow generator 108 through the inlet conduit 110. The gas can be filtered through a filter 111 and delivered to the humidifier 104 through a humidifier inlet 114. The gas is humidified as it passes through a humidifier chamber 116, and the gas flows out through a humidifier outlet 118 and into the patient conduit 112. The gas then moves through the patient conduit 112 to the patient 102 via a connector 140 and the patient interface 136. The patient interface 136 may be, for example, but not limited to, a surgical access device such as a trocar or cannula for laparoscopic surgery. In some examples, the flow of gases out of the patient interface 136 may be diffused. It will be appreciated that other surgical access devices may be utilized to deliver and/or remove gases from the surgical cavity and throughout the disclosure the term cannula is used as an example of a surgical access device.

The humidifier chamber 116 may be removably engageable with a body 124 of the humidifier 104. The humidification chamber 116 may comprise a side wall, and a base/bottom. The humidification chamber 116 may further comprise a top, an openable lid, or may be topless (e.g. open topped, in which case, the humidification chamber 116 may be received in a sealable cavity of the humidifier body 124). The top, base and side walls may together define a substantially circular chamber when the chamber is viewed from the top. The side wall is arcuate in shape and may define other compound arcuate shapes. For example, the chamber may be an elliptical chamber when viewed from the top, or any other chamber having an arcuate but not necessarily perfectly circular shape when viewed from above. The chamber may be symmetrical about at least one axis when viewed from the top. The chamber may be symmetrical about 2 axes, that is, a horizontal and a vertical axis when viewed from above. In some examples the humidification chamber may be refillable via a water source, for example a water bag. The humidification chamber may comprise one or more float assemblies to allow for flow of water from the water source when the water level in the chamber is below a threshold.

As shown in for example FIG. 1, the humidifier 104 has a heater plate 122. The base 121 of the humidification chamber 116 may have a heat conductive (e.g. metal) region or may be entirely heat conductive, and may be adapted to hold a volume or reservoir of water 120, which can be heated by a heater plate 122. The heater plate 122 may be in thermal contact with the heat conductive base 121 of the humidification chamber 116. Providing power to the heater plate 122 can cause heat to flow from the heater plate 122 to the water 120 through the heat conductive base 121. As the water 120 within the humidification chamber 116 is heated it can evaporate and the evaporated water can mix with gases flowing through the humidification chamber 116 from the flow generator 108, optionally via the filter 111 (for example pass-over humidification).

The humidified gases leave the humidifier via outlet 118 (which in the example of FIG. 1 is an outlet of the humidification chamber 116) and are passed to the patient 102 via the patient conduit 112, the connector 140, the patient interface 136 and into the surgical site to, for example, insufflate the surgical site and/or expand a body cavity.

In some examples, the humidifier 104 may include a gases heater and separate humidification part of the humidifier 104 which humidifies the gases. Each of the gases heater and the humidification part may have separate heaters. In these examples the gases heater may be configured to provide heat to the gases flow (optionally in addition to that provided by the humidification part of the humidifier) while the humidification part of the humidifier is configured to humidify the gases flow. The conduit heating wire 134 may also aid in heating the gases (or at least maintaining the temperature of the gases) as described in more detail below.

The humidifier may be controlled by a specific humidifier controller (for example a humidifier control system 106) and alternatively or additionally, by a system controller that may be located elsewhere in the system (for example as part of the flow generator).

The humidifier control system 106 is configured to control a temperature and/or humidity of the gases being delivered to the patient 102. The humidifier control system 106 can be configured to regulate an amount of humidity supplied to the gases by controlling an electrical power supplied to the heater plate 122.

The humidifier control system 106 can control operation of the humidifier 104 in accordance with instructions set in software and in response to sensors (for example as outputs of the sensors described in more detail below).

The system may comprise one or more sensors, which generate sensor outputs. The sensor outputs may be indicative of a property which the sensor is configured to measure. In some examples, the sensors may be configured to measure a gas property (for example temperature, humidity, pressure, flow rate, gas composition etc). In some examples, the sensors may be configured to measure a property of a component of the system (for example a heater temperature, or a current or power provided to the heater).

The one or more sensors may also comprise at least one patient monitoring sensor. In some examples the patient monitoring sensors may comprise a patient core body temperature sensor, an external patient temperature sensor and/or a heart electrical activity sensor (for example a heart rate detector and/or an electrocardiogram (ECG), a blood pressure sensor, a blood gas sensor (for example CO₂ or O₂), a breathing gas sensor (for example CO₂ or O₂). The system may use the output of the patient monitoring sensor(s) to determine the desired dew point and/or desired temperature of the gases. Determining the desired dew point and/or desired temperature of the gases based on an output of the output of the patient monitoring sensor(s) may allow for the system to determine how much energy to transfer to patient based on for example external patient temperature and/or patient core body temperature. In some examples the desired dew point and/or desired temperature of the gases is based on the external patient temperature and/or patient core temperature. In some examples a temperature of the gases as they exit the surgical cavity (for example at or near the cannula) may be used to determine a patient temperature.

In some examples a temperature of the gases as they exit the surgical cavity (for example at or near the cannula) may be used to determine whether or not, or how much energy to transfer to the patient. In some examples, one or more operating parameters of the system (for example dew point, flow rate and/or gases temp) may be based, at least in part on, the temperature of the gases as they exit the surgical cavity.

In some examples external patient temperature and/or patient core body temperature is monitored before, during and after the surgical procedure.

The sensor(s) may be connected to the controller via a wired connection, or be wirelessly connected to the controller. In some examples, the sensor(s) may be connected to the controller by a lead. In some examples, the sensor(s) are connected to the controller by integrated wires rather than an external cable.

The sensor(s) may be integrated with a component, or connectable and disconnectable (for example provided to a conduit via a port).

Examples of various sensors are provided below. It is appreciated that these examples could be combined. For example, the humidifier control system 106 can receive temperature information from the heater plate sensor 126 which it can use as an input to a control module used to control the power or temperature to a set point of the heater plate 122. The humidifier control system 106 can be provided with inputs of temperature and/or flow rates of the gases. In some examples, the chamber outlet temperature sensor 128 can be provided to indicate to the humidifier control system 106 the temperature of the humidified gases as they leave the outlet 118 of the humidification chamber 116. The temperature of the gases exiting the chamber can be measured using any suitable temperature sensor 128, such as a wire-based temperature sensor. In some examples, a chamber outlet flow sensor 130 may be provided to indicate to the humidifier control system 106 the flow rate of the humidified gases. The flow rate of the gases through the chamber 116 can be measured using any suitable flow sensor 130, such as a hot wire anemometer, differential pressure flow sensors, ultrasonic flow sensors, or a thermistor configured for use as a flow sensor. In some examples, the temperature sensor 128 and flow sensor 130 are in or otherwise provided to the same sensor housing. The temperature sensor 128 and flow sensor 130 can be connected to the humidifier 104 via connector 132. Additional sensors may be incorporated into the system 100, for example, for sensing parameters at the patient end of the patient conduit 112.

The system may comprise at least one temperature sensor. The at least one temperature sensor may be configured to measure a temperature of the gases flow. The at least one temperature sensor may be located in a gases flow path, at any one or any combination of: at or near an end of an outlet and/or inlet of the humidifier, at or near an end of a conduit connected to the humidifier(i.e. a humidifier end of a patient conduit 112—for example temperature sensor 128), at or near an end of a conduit connected to the patient interface (i.e. a patient end of a patient conduit 112), at or near an inlet of a patient interface 136 (for example a cannula) which is configured to deliver the gases flow to the surgical site, at or near an outlet of a patient interface 136 (for example a cannula) which is configured to deliver the gases flow to the surgical site, in the flow generator. In some examples the system may comprise an ambient temperature sensor. The ambient temperature sensor may be used, for example, in controlling one or more temperature set points of the system (for example dew point and/or gases temperature).

The system may comprise at least one flow rate sensor. The at least one flow rate sensor being configured to measure a flow rate of the gases flow. The at least one flow rate sensor may located in a gases flow path, at any one or any combination of: at or near an end of an outlet and/or inlet of the humidifier, at or near an end of a conduit connected to the humidifier (i.e. a humidifier end of a patient conduit 112—for example flow rate sensor 130), at or near an end of a conduit connected to the patient interface (i.e. a patient end of a patient conduit 112), at or near an inlet of a patient interface 136 (for example a cannula) which is configured to deliver the gases flow to the surgical site, at or near an outlet of a patient interface 136 (for example a cannula) which is configured to deliver the gases flow to the surgical site, in the flow generator.

In some configurations the flow rate may be determined indirectly based on the output of one or more other sensors. The flow rate may be determined by, for example, one or more electrical characteristic of one or more heaters in the system (for example the heater plate, conduit heater, or heater element of the filter). The one or more electrical characteristics may be for example current, power drawn, duty cycle.

In some examples, the system may comprise at least one pressure sensor. The at least one pressure sensor may be configured to measure a pressure of the gases flow. The at least one pressure sensor may be located in a gases flow path, at any one or any combination of: at or near an end of an outlet and/or inlet of the humidifier, at or near an end of a conduit connected to the humidifier, at or near an end of a conduit connected to the patient interface, at or near an inlet of a patient interface 136 (for example a cannula) which is configured to deliver the gases flow to the surgical site, at or near an outlet of a patient interface 136 (for example a cannula) which is configured to deliver the gases flow to the surgical site, in the flow generator.

The at least one pressure sensor may be used to measure the pressure of the surgical cavity either directly (via a pressure sensor located in or near the surgical cavity i.e. on a cannula and/or medical instrument inserted into the surgical cavity), or indirectly via measurement of the gases flow into or out of the surgical cavity.

In some examples, the system may comprise at least one humidity sensor. The at least one humidity sensor may be configured to measure a humidity of the gases flow. The at least one humidity sensor is located in a gases flow path, at any one or any combination of: at or near an end of an outlet and/or inlet of the humidifier, at or near an end of a conduit connected to the humidifier, at or near an end of a conduit connected to the patient interface, at or near an inlet of patient interface 136 (for example a cannula) which is configured to deliver the gases flow to the surgical site, at or near an outlet of patient interface 136 (for example a cannula) which is configured to deliver the gases flow to the surgical site, in the flow generator.

Core body temperature may be measured by one or more methods for example by measurement rectally, within the surgical cavity, nasally, esophageal, ear (tympanic), bladder, and/or a temperature of a surface of the surgical cavity. In some examples one or more core body temperature sensors may be provided as part of the system to measure core body temperature. The core body temperature sensor may be located at one of the above locations to measure core body temperature.

As described above core body temperature may be measured by a sensor located in the surgical cavity. The core body temperature sensor may be a probe or other locatable sensor, or be located in, or part, of the cannula. In some examples, the core body temperature sensor is located at the distal end of cannula. In some examples, the core body temperature sensor is located on a medical instrument inserted into the surgical cavity. In some examples, the core body temperature sensor contacts a patient tissue in the surgical cavity.

In some examples, the system may comprise an external patient temperature sensor. The external patient temperature sensor may be configured to measure an external temperature of the patient, for example a skin temperature. The external patient temperature can have a relationship to a core patient temperature. In some examples, the temperature sensor may be placed on the patient's skin, for example by adhesive.

The external patient temperature and/or patient core body temperature may be used by the system to control, at least in part, a desired dew point and/or desired temperature of the gases and/or desired flow rate of the gases. The external patient temperature and/or patient core body temperature may also be used to determine a desired heat transfer, which then can be used to determine a desired dew point and/or desired temperature of the gases and/or desired flow rate of the gases.

In some examples, the external patient temperature and/or patient core body temperature may be displayed to the clinician (for example via a user interface) and the clinician may make therapy based decisions (for example to increase, decrease or maintain the heating provided to the patient). The system may, based on input from the clinician and the external patient temperature and/or patient core body temperature, control a desired dew point and/or desired temperature of the gases and/or desired flow rate of the gases. In some examples, the clinician may input the operating parameters via a user interface (for example a desired dew point and/or desired temperature of the gases and/or desired flow rate of the gases).

As described above one or more sensors may be located in the patient interface 136 (for example a cannula). The sensors may be incorporated into the cannula as for example as described in, WO2020201946, which is incorporated by reference herein in its entirety.

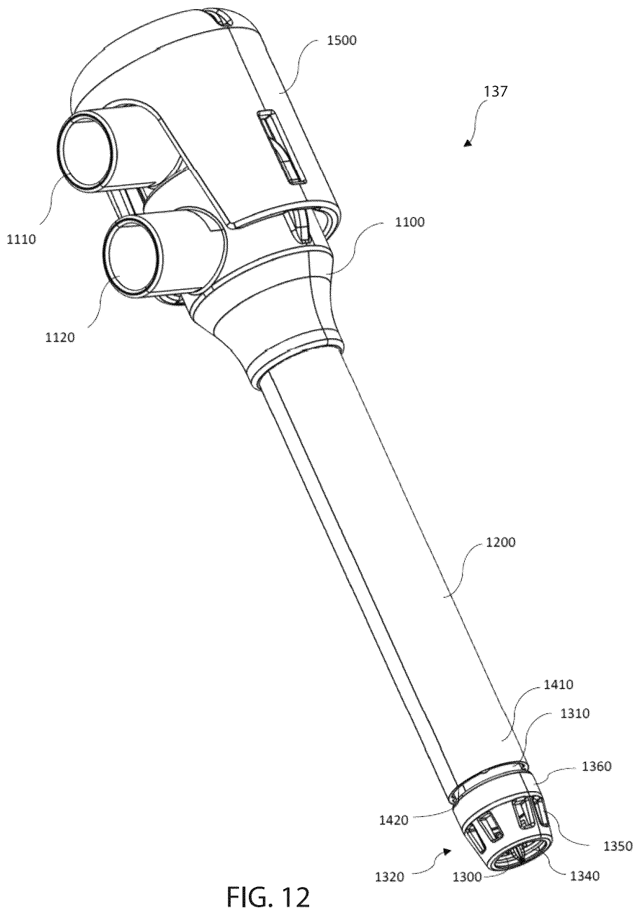

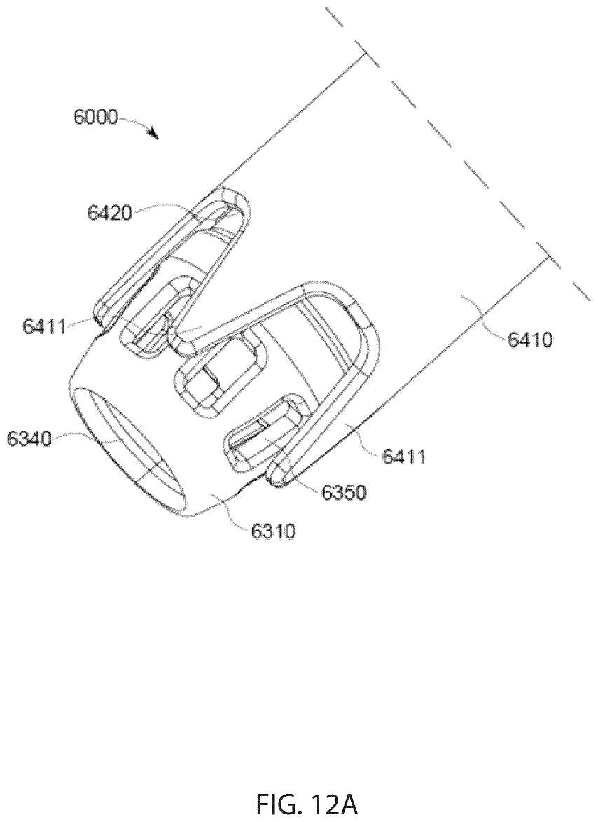

For example, the cannula may comprise a sensor located in, or in communication with the gases flow path of the cannula. The cannula may also include a pressure channel which is in fluid communication with the gases flow path of the cannula, and the pressure sensor is configured to measure pressure in the pressure channel. The pressure sensing channel may be located along portions of shaft 1200 for example as shown in FIG. 12.

In some examples, the one or more sensors may be provided as one or more probes which extend outside the cannula through one or more apertures of the cannula and optionally into a gases pathway of the cannula or the surgical cavity. The aperture may comprise one or more seals.

The humidifier control system 106 can control the humidifier to humidify the gases to a desired dew point. For example, the humidifier control system 106 can control a dew point of the gases to the desired dew point. The desired dew point may be a dew point at or near an outlet of a patient interface 136 (for example a cannula) which is configured to deliver the gases flow to the surgical site. In some examples, the desired dew point may be a dew point at or near an end of an outlet and/or inlet of the humidifier, at or near an end of a conduit connected to the patient interface (i.e. a patient end of a patient conduit 112), an end of a conduit connected to the humidifier (i.e. a humidifier end of a patient conduit 112), or at or near an inlet of a patient interface 136 (for example a cannula) which is configured to deliver the gases flow to the surgical site.

The dew point of the gases may be determined based on the output of one or more sensors. The humidifier control system 106 may run an algorithm that determines dew point of the gases based on the output of one or more sensors (for example heater plate temperature, flow rate, and a humidifier outlet temperature (i.e. a chamber outlet temperature), ambient temperature, and/or device temperature). In some examples, the dew point of the gases may be determined based on at least the output of at least one temperature sensor, and/or at least one flow rate sensor. In some examples, the dew point of the gases may be estimated based on at least the output of at least one temperature sensor, and/or at least one flow rate sensor.

In some examples, the sensor(s) described above may be located in the patient interface 136 (for example at or near an inlet of a patient interface 136, and/or at or near an outlet of a patient interface 136.)

The humidifier control system 106 may also control the humidifier to heat the gases flow to a desired temperature. Heat may be provided to the gases flow from evaporation of the water, and/or heat transfer from the water in the humidification chamber 116. In some examples, the humidifier 104 may be used in addition to the conduit heater 134 to heat the gases to a desired temperature.

The humidifier control system 106 can be in communication with the heater of the humidifier (for example heater plate 122) such that the humidifier control system 106 can control the heater of the humidifier 104. The humidifier control system 106 may control for example a power delivered to the heater plate 122. In some examples, the humidifier control system 106 may control the heater to a temperature set point. As described further herein, the humidifier control system 106 can determine an amount of power to deliver to the heater plate 122, or a heater plate set point, based at least in part on any one or more of a flow condition, an operation mode, a flow reading, an outlet temperature reading, a heater plate sensor reading. The heater plate temperature set point and/or heater plate power may be based on a desired dew point.

The system 100 can include a conduit heater (for example a conduit heating wire 134) configured to provide heat to the gases traveling along the patient or gas delivery conduit 112. Gases leaving the outlet 118 of the humidification chamber 116 can have a high relative humidity (e.g., about 100%). As the gases travel along the patient conduit 112 there is a chance that water vapor may condense on the conduit wall, reducing the water content of the gases. To reduce condensation of the gases within the conduit, the conduit heating wire 134 can be provided within, throughout, and/or around the patient conduit 112, including being at least partly within a wall forming the patient conduit 112.

One or more conduits in the system may be heated, for example, the patient conduit 112, inlet conduit 110, recirculation return conduit 211, recirculation supply conduit 210, the conduit between the secondary flow generator 150 and humidifier, 104, and/or any other conduit in the system.

One or more conduits may be provided with a conduit heater to heat the gases flowing through the conduit. The conduit may be for example a heater wire as described elsewhere in the specification.

Each of the conduit heaters may be independently controlled. In some examples the conduits may be controlled in control groups. The conduit heaters may be controlled by a controller of the flow generator 108, secondary flow generator 150, and/or humidifier 104.

The conduit heating wire 134 may be used in addition to, or alternatively to the humidifier to heat the gases to a desired temperature.

The system may control a temperature of the gases to the desired temperature. The temperature of the gases may be based on the output of one or more sensors (for example the temperature sensors as described in more detail elsewhere in the specification).

The desired temperature may be a temperature at or near an outlet of a cannula which is configured to deliver the gases flow to the surgical site. In some examples, the desired temperature may be a temperature at or near an end of an outlet and/or inlet of the humidifier, at or near an end of a conduit connected to the patient interface (i.e. a patient end of a patient conduit 112), an end of a conduit connected to the humidifier, (i.e. a humidifier end of a patient conduit 112), at or near an inlet of a patient interface 136 (for example a cannula) which is configured to deliver the gases flow to the surgical site, or in the surgical cavity.

Power can be supplied to the conduit heating wire 134 from the humidifier 104 and can be controlled through the humidifier control system 106. In some embodiments, the heating wire 134 is configured to maintain the temperature of the gas flowing through the patient conduit 112. In some embodiments, the conduit heating wire 134 can be configured to provide additional heating of the gases to elevate the gases temperature.

The gas delivery conduit may be single walled or comprise a dual conduit configuration that includes an outer conduit and an inner conduit. The inner conduit may carry the gases and include the heating wire 134. The outer conduit may provide insulation of the inner conduit. The outer conduit and inner conduit may be co-axial. There may be an air gap between the outer conduit and the inner conduit.

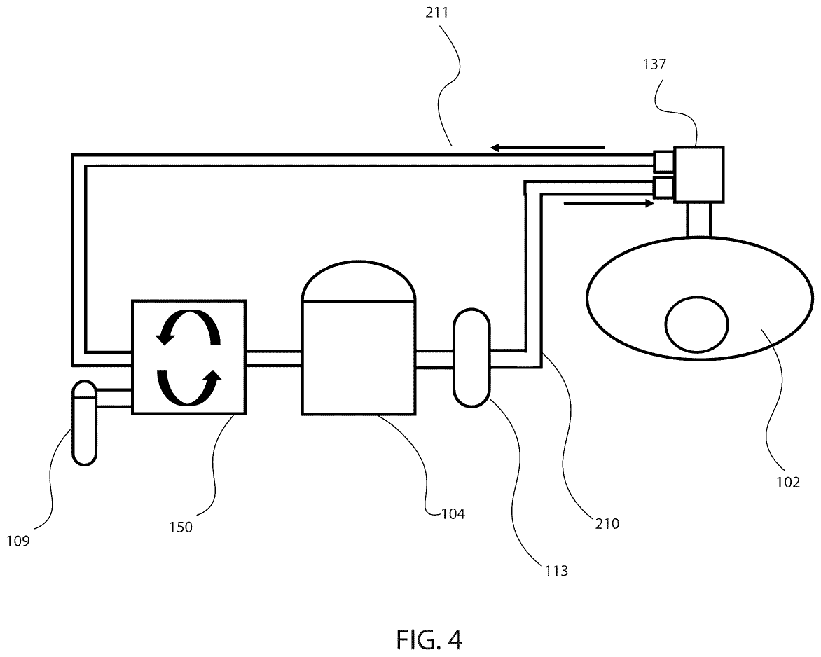

Additionally or alternatively, a filter or filter assembly may be optionally provided and disposed in use between the humidifier 104 and the patient interface 136 or recirculation cannula 137 (for example as shown as filter 113 in FIGS. 2 to 6). The filter may provide filtering of the gases before delivery to the patient. In some examples, the filters may allow re-use of the inlet conduit 110 and/or an inspiratory conduit, and in some instances re-use of the humidification chamber 116. The filter 113 may be provided for example at an outlet of the humidifier (see for example FIG. 3), inline with the supply conduit 210 (see for example FIGS. 4-6), or at or near the cannula or patient interface.

In some configurations a filter or filter assembly 115 may be provided downstream of the recirculation cannula 137 (for example cannula 137 or outlet cannula 148). In some configurations the filter 115 may be positioned between the patient interface 136 or recirculation cannula 137 and the recirculation flow generator 150.

In some configurations a filter or filter assembly 115 may be provided between a recirculation flow generator 150 and a recirculation cannula 137 to filter gases from the surgical cavity.

The filter 115 may be provided for example at an outlet of the flow generator, inline with the recirculation return conduit 211, or at or near the cannula or patient interface.

Additionally, or alternatively, in some configurations, a filter or filter assembly may be provided upstream of the recirculation flow generator 150 and/or between the recirculation flow generator 150 and the outlet of the patient cavity (i.e. recirculation cannula 137 and/or outlet cannula 148) and/or at an inlet of the recirculation flow generator 150.

The filter assembly 111, 113, 115, 181 can comprise a filter medium for filtering the gases. The filter assembly may also comprise a housing and a heating element. The filter medium can be positioned inside the housing so that the humidified gases flowing through the housing are filtered and particles removed. The heating element can be positioned in the gases flow path between the inlet and the outlet of the housing. The heating element may be spaced apart from the filter medium and/or the housing. The heating element can be configured to heat the filter medium to reduce condensation and prevent the filter becoming clogged. Such filters are described in, for example, WO2018/106127, which is incorporated by reference herein in its entirety.

The filter assembly may be integrated as part of the humidifier and/or flow generator. The filter assembly may be user replaceable. In some examples, the filter assembly may be a separate component which is fitted as part of the recirculation circuit, and/or connected to a conduit of the system.

The filter assembly 111, 113, 115, 181 may be heated for example to help to prevent or minimize condensation or evaporate moisture which has been absorbed by the filter medium. This may be of importance in a recirculation system as humidified gases are being passed through the filter. The filter assembly 111, 113, 115, 181 may be heated by the same heater as an attached conduit, or a separate heater.

In some examples, the suction source 141 may have a filter, or a filter located may be in pneumatic connection with, and/or located upstream of the suction source 141.

Components that make up the gases flow path in the system (for example the conduits and humidifier) may be configured to be connectable and disconnectable from other components. The components may have complementary connectors.

In some examples, the humidifier chamber 116 of the humidifier 104 can be configured to provide a controlled gases flow pattern for the insufflation gases. The humidification chamber 116 may be that as disclosed in WO2019190332, which is incorporated by reference herein in its entirety. The humidification chamber 116 can be configured to cause the gases entering the humidification chamber 116 via the inlet 114 to swirl in a vortex (i.e. spiral) within the chamber 116 as the gases exit the chamber 116 via the outlet 118. For example, the inlet 114 may be provided in the side wall and orientated relative to the side wall such that the gases flow enters the humidification chamber 116 via the inlet 114 at a direction substantially tangential to the side wall. In other words, the inlet 114 may be positioned on the humidification chamber 116 and orientated relative to the side wall such that the gases flow along at least a portion of the internal surface of the side wall when entering the humidification chamber 116. Having the gases swirling in a vortex i.e. spiral within the humidification chamber 116 improves the performance of the humidifier 104, and by extension of the system 100, by increasing the efficiency of the humidification chamber 116. Performance and/or efficiency of the chamber may be increased by increasing the residence time of the gases in the chamber and/or increasing the gases flow path length.)

In some examples, the inlet 114 may be provided in the side wall of the chamber 116, with the inlet 114 configured such that the direction of gases flow as that flow exits the inlet 114 and enters the chamber 116 is not aligned perpendicular to the side wall. The inlet 114 is arranged such that the gases are introduced substantially tangential to a side wall such that the gases introduced into the chamber attach to the wall of the chamber and travel around the chamber to create a spiral flow. In other embodiments, the inlet 114 may be provided fully in the side wall of the chamber 116.

Additionally, and/or alternatively, the inlet 114 may be angled (e.g. angled down towards the bottom of the humidification chamber 116) such that the gases entering the humidification chamber 116 at a direction substantially tangential to the side wall are guided towards the water 120.

The system 100 may include a gases recirculation circuit which comprises a recirculation flow generator 150 configured to recirculate gases from the surgical cavity of the patient through a humidifier and back to the surgical cavity of the patient.

The gases recirculation circuit allows for high humidity gases to be provided to the surgical cavity (i.e. gases above a dew point as described in more detail below).

As described above gases recirculated from the surgical cavity may already have already been previously heated and humidified, and so less energy needs to be expended by the humidifier to heat and humidify the gases to the desired temperature and dew point.

Further, the recirculation circuit may have one or more condensate management systems (as described in more detail below) to reduce the formation of condensate in the circuit (for example via heating and/or insulation of the circuit) and to reduce the amount of humidity lost from the circuit (for example by reevaporating any condensate). The one or more condensate management systems may help to help maintain humidity in the flow of gases recirculation circuit and reduce the amount of humidification fluid (i.e. water) which needs to be added to the gases.

Further, recirculation of the gases may use less water, as the amount of water vapour which needs to be added to the gases is less than if the gases were fresh (i.e. comparatively low humidity). This may mean that the chamber 116 lasts longer between refills and/or needs to be refilled less frequently. It may also mean that a chamber with a lower humidification fluid (i.e. water) capacity or volume can be used.

Recirculation of gases may be of particular importance, when targeting higher dew points. When the gases are recirculated, humidity only needs to be added to the extent it was lost during delivery (for example as condensation in the surgical cavity, or condensation in the system), diluted due to addition of further fresh gases (for example additional gases added to the surgical cavity), or lost due to venting for example via smoke evacuation or other system leaks. This decreases the demand placed on the humidifier to provide humidity to the gases as the humidifier is adding humidity to already humidified gases rather than to relatively dry fresh gases. Recirculation also decreases the amount of humidification fluid (i.e. water) needed to humidify the gases. Recirculation of gases may also reduce the demand for further fresh gases (for example help to conserve gases). Reducing the demand for further fresh gas/conserving gases may be of further importance when high flow rates of gases are provided, for both delivery of therapy and/or smoke clearance.

FIG. 2 and FIG. 3 show examples of a system 100 comprising a primary flow generator 108 and a recirculation flow generator 150. The system 100 comprises a recirculation circuit comprising the recirculation flow generator 150 and a humidifier 104.

The recirculation flow generator 150 and humidifier 104 may be in separate apparatuses. In some examples, the recirculation flow generator 150 and humidifier 104 may be integrated (for example into a common housing).

The recirculation flow generator 150 is configured to recirculate gases from the surgical cavity of the patient through a humidifier and back to the surgical cavity of the patient.

In some examples, the recirculation flow generator 150 comprises a valving arrangement and is configured to control the flow of gases by a valving arrangement.

In some examples, the recirculation flow generator 150 comprises a blower and is configured to generate the flow of gases by the blower. The blower may be controlled to generate the flow of gases at the desired flow rate and/or the desired pressure.

In some cases, both a valving arrangement and a blower may be used.

In some examples, the flow generator comprises a pump. The pump may be a diaphragm compressor, peristaltic pump, radial compressor, rotor system, or other type of suitable pump.

The recirculation flow generator 150 may be single use (i.e. being disposable after use, or able to be sterilized between patients. In some examples, the recirculation flow generator 150 may be able to be reused by having suitable filters before and optionally after the recirculation flow generator 150, and/or by replacement or sterilization of the remainder of the system between uses.

In some examples a portion of the recirculation flow generator 150 is single use or able to be sterilized between patients. For example, the recirculation flow generator 150 may comprise a recirculation flow generator portion which is replaceable or able to be sterilized. The recirculation flow generator portion may comprise at least part of, or all of the gases flow path of the recirculation flow generator 150.

The recirculation flow generator portion may be provided as a removable and replaceable cartridge. In some examples portions of the flow generator recirculation flow generator portion comprises all of the components of the recirculation flow generator 150 which come into contact with the gases. The recirculation flow generator portion may be decoupled from the motor (or other operating component).

Use of a disposable cartridge that includes for example, in a flow generator with a diaphragm compressor, a chamber, inlet, outlet and diaphragm allows for simple and cost effective single use design, and replacement of all components of the recirculation flow generator 150 which come into contact with the gases.

The recirculation flow generator 150 may comprise or be connected to one or more check valves to ensure that gases flow in the desired direction and/or do not flow in the incorrect direction in portions of the circuit. The check valves may be provided to the inlet and/or outlet of the recirculation flow generator, or placed in positions in the recirculation circuit.

It will be appreciated that features of the recirculation flow generator may be equally applicable to features of the primary flow generator.

The recirculation flow generator 150 may comprise a recirculation inlet to receive gases from the surgical cavity of the patient (for example from the outlet port of the cannula 137). The recirculation flow generator 150 may comprise a gases source inlet (for example as described in more detail below with respect to FIG. 4.) The gases source 109 may be pressurised.

In some examples, the recirculation flow generator 150 and the humidifier 104 may be separate apparatuses, or be combined to form a single apparatus or the two separate components may be modular and configured to couple together without the need for separate conduit(s) therebetween, as is known in the art.

The system may comprise at least one recirculation cannula 137. The at least one recirculation cannula 137 may be a single recirculation cannula 137, as shown in FIG. 2, FIG. 3, FIG. 4, and FIG. 12, which provides two gases pathways, an outlet gas pathway (for example an outlet lumen) providing a pathway for gases from the surgical cavity and an inlet gas pathway (for example a delivery lumen) for providing a pathway for gases to the surgical cavity. The recirculation cannula 137 may comprise an outlet (for example an outlet port) connected to the outlet gas pathway, and an inlet (for example a delivery gas inlet port) connected to the inlet gas pathway.

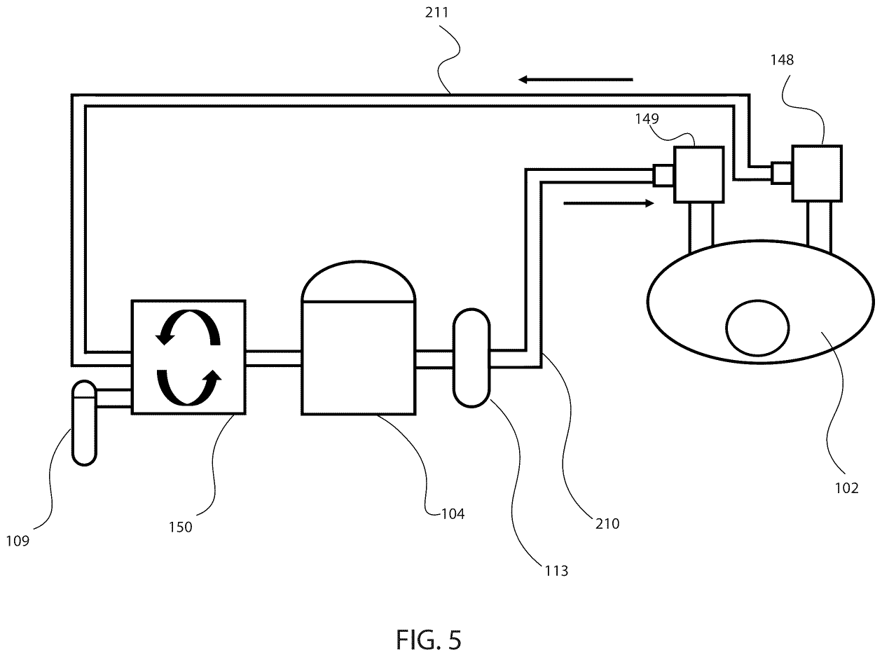

In some examples, the system, for example as shown in FIG. 5, may have two recirculation cannulas, with an outlet cannula 148 providing an outlet gases pathway for gases from the surgical cavity and an inlet cannula 149 providing an inlet gases pathway to the surgical cavity. In some configurations the inlet cannula and/or the outlet cannula may be the cannula 137 which provides two gases pathway as described elsewhere in the specification. In systems where the cannula 137 is used, the inlet or outlet of the cannula 137 which is not used as part of the recirculation circuit may be connected to another component or device. For example, the outlet of the cannula 137 which is not in use may be used as a venting cannula to vent smoke via a filter, and/or connected to a vacuum or suction source.

In some examples, the flow of gases out of the patient interface 136 may be diffused. This may help distribute the gases within the cavity. Distributing or diffusing the gases within the surgical cavity may avoid or reduce jetting of gases at a tissue surface.

FIG. 12 shows an example of a cannula 137. The cannula may be that as described in (PCT/IB2023/063152) which is hereby incorporated by reference. The cannula 137 comprises a cannula housing 1100 and a cannula shaft 1200 extending from the housing 1100. The cannula shaft 1200 defines a delivery lumen 1300, which is configured to deliver fluid flow to the body cavity of a patient during a surgical procedure. In the illustrated examples, the cannula shaft 1200 includes a delivery shaft 1310, which defines the delivery lumen 1300. As illustrated, the delivery shaft 1310 comprises an elongate cylindrical tube, although non-cylindrical shapes and/or noncircular cross-sections are also contemplated.

The delivery lumen 1300 can be provided in fluid communication with a delivery inlet. In the illustrated examples, the delivery inlet is provided in the form of delivery inlet port 1110 in the cannula housing 1100. The delivery inlet port 1110 can be configured for connection to a fluid source. For example, the delivery inlet port 1110 may be connectable to a fluid delivery tube in fluid communication with the fluid source to facilitate fluid flow from the fluid source into the delivery lumen 1300. The delivery inlet port 1110 may connect to a fluid delivery tube via a press-fit connection (such as a taper fit, for example), although other suitable connection types may also be used. In some examples the fluid delivery tube may be permanently connected to the delivery inlet port 1110.

The cannula shaft 1200 includes an outlet shaft 1410, which defines the outlet lumen (as an outlet gas pathway). The outlet shaft 1410 comprises an elongate cylindrical tube, although non-cylindrical shapes are also contemplated. The outlet lumen provides a fluid flow path between an inlet 1420, for example a venting inlet, at or adjacent to a distal end of the outlet.

The outlet can be in the form of an outlet port 1120 in the cannula housing 1100. The outlet port 1120 may be configured for connection to a venting element, such as a suction source and/or suction canister, which may be provided in connection with a filter.

When the cannula is used in the systems as the recirculation cannula 137 for example as shown in FIGS. 2, 3, 4, 8 and 9 the inlet can be connected to the recirculation supply conduit 210, and the outlet connected to the recirculation return conduit 211.

In the example cannula of FIG. 12, the delivery outlet 1320 comprises an open distal end 1340 of the delivery lumen and a plurality of diffusion apertures 1350. The diffusion apertures 1350 may be provided through a side wall of the delivery shaft 1310 and in fluid communication with the delivery lumen 1300. The diffusion apertures 1350 may be configured to diffuse at least a portion of the fluid flow that exits the delivery lumen 1300 into the body cavity. The delivery outlet 1320 may be configured such that fluid flows from the delivery lumen 1300 into the body cavity through both the open distal end 1340 and through the diffusion apertures 1350.

The diffusion apertures 1350 may be configured to diffuse fluid flow around the cannula shaft and/or past a distal end of the cannula shaft. The diffusion apertures 1350 may be spaced around a diameter of the delivery shaft 1310. In some examples, the diffusion apertures 1350 may be spaced uniformly (or substantially uniformly) around the delivery shaft 1310. For example, the diffusion apertures 1350 may be arranged around the delivery shaft 1310 with rotational symmetry. Substantially uniform and/or rotationally symmetrical spacing of the diffusion apertures 1350 may contribute to substantially even distribution of fluid flow to the body cavity of the patient.

In some embodiments, the apertures 1350 may be arranged in pairs. The pairs of apertures may be uniformly spaced around the delivery shaft 1310, for example. In other examples, the diffusion apertures can be spaced non-uniformly around a diameter of the cannula shaft 1200.

The cannula 137 may comprise a proximal end housing 1500 attachable to the cannula 1000. The proximal end housing 1500 may be attachable to the cannula housing, 1100, for example. The proximal end housing 1500 may cover a portion of the cannula housing 1100, in the manner of a cap.

The cannula 137 can comprise one or more seals. The one or more seals may be configured to prevent or reduce fluid flow from exiting the cannula shaft 1200 other than through the delivery outlet and/or the outlet port 1120. For example, one or more seals may be configured to seal a proximal end of the cannula 1000 to prevent fluid flow from escaping at a proximal end of the cannula. For example, there may be sealing in the proximal end of the cannula 137. One or more seals may be further configured to prevent entrainment of fluid (such as air) into the cannula and/or into a venting tube. This may be particularly important in cases where the cannula 137 is used as part of a recirculation system in which vented gases are re-introduced into the body cavity of the patient.

Sealing of the recirculation circuit may help to prevent entrainment of ambient air, ensure that pressure in the surgical cavity is maintained, and the volume of the surgical cavity is maintained.

The cannula 137 can further comprise an instrument seal, for example. The instrument seal may be configured to provide a substantially fluid tight seal between the cannula housing 1100 and a medical instrument, when a medical instrument is received in the cannula 137. The instrument seal may define an aperture for receiving a medical instrument therethrough.

Cannula 137 may include one or more structures configured to inhibit withdrawal of the cannula from the body cavity of the patient. For example, the cannula may include one or more securement components configured for attaching and/or securing the cannula to the patient, or securing the cannula in position in an access incision. A securement component may be configured to be positioned within the body cavity and/or tissue of the patient, and/or external to the body cavity of the patient.

For example, cannula 137 may include, or be configured to connect with, one ore more suture tabs. A suture tab may accommodate one or more sutures for securing the cannula to patient to minimize risk of the cannula being inadvertently withdrawn or partly pulled out from, the surgical cavity. Reducing risk of withdrawal of the cannula can assist to inhibit or reduce risk of entrainment of fluid, for example air, from external to the body cavity in the event the canula is partially withdrawn from the body cavity. A securement component may assist to keep at least a venting inlet, such as inlet 1420, located within the body cavity while recirculation flow generator 150 is in operation.

In some examples, the cannula may include one or more projections at a distal end of the cannula shaft. The one or more projections may be configured to inhibit or minimize entrainment of fluid, for example air, from external to the body cavity, as may happen if the cannula is partially withdrawn from the body cavity, such that venting inlet 1420 is at least partly out of the body cavity.

One example of a cannula including projections 6411 is shown in FIG. 12A. In this example, the outlet shaft 6410, which may function as a venting shaft, includes distally extending projections 6411 at the distal end of the outlet shaft 6410, adjacent venting inlet 6420. As shown in the example of FIG. 22, the projections 6411 have a tapered shape, narrowing as the projections 6411 extend towards the distal end of the cannula. Other shapes of the projections 6411 are also contemplated. The projections 6411 may be spaced apart from a distal end of the delivery shaft 6310. The projections 6411 may be flush with a distal end of the delivery shaft 6310.

The projections 6411 may be configured to minimise impedance of fluid delivery. For example, the projections 6411 may be positioned to minimise overlapping with the diffusion apertures 6350. In the example of FIG. 22, the projections 6411 are positioned between adjacent diffusion apertures 6350. The projections 6411 may serve to decrease an exposed area of the distal end of the delivery shaft 6310. This may assist in mitigating risk of entrainment of air from outside the body cavity into the venting inlet 6420 should the cannula 6000 be withdrawn, or dislodged from the body cavity or surrounding tissue of the patient. In the illustrated example, the cannula 6000 includes an open distal end 6340. However, in other examples, the cannula 6000 may include a blind distal end.

An outlet of the humidifier 104 is pneumatically connected to the recirculation cannula 137 (for example an inlet of the cannula 137 or inlet cannula 149) by a recirculation supply conduit 210. The recirculation flow generator 150 is pneumatically connected to the recirculation cannula (for example an outlet of the cannula 137 or an outlet cannula 148) by a recirculation return conduit 211.

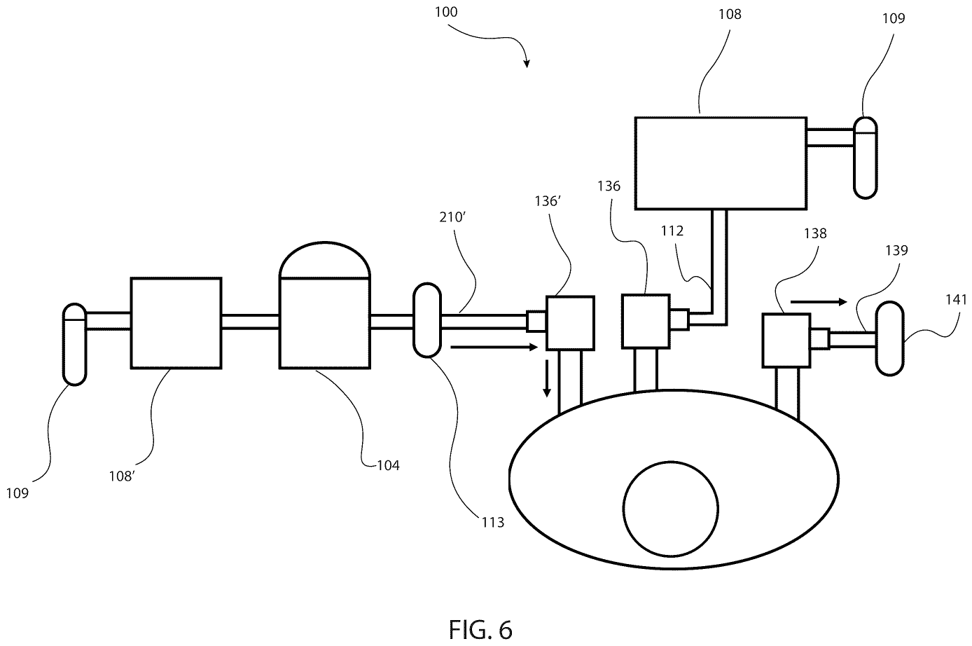

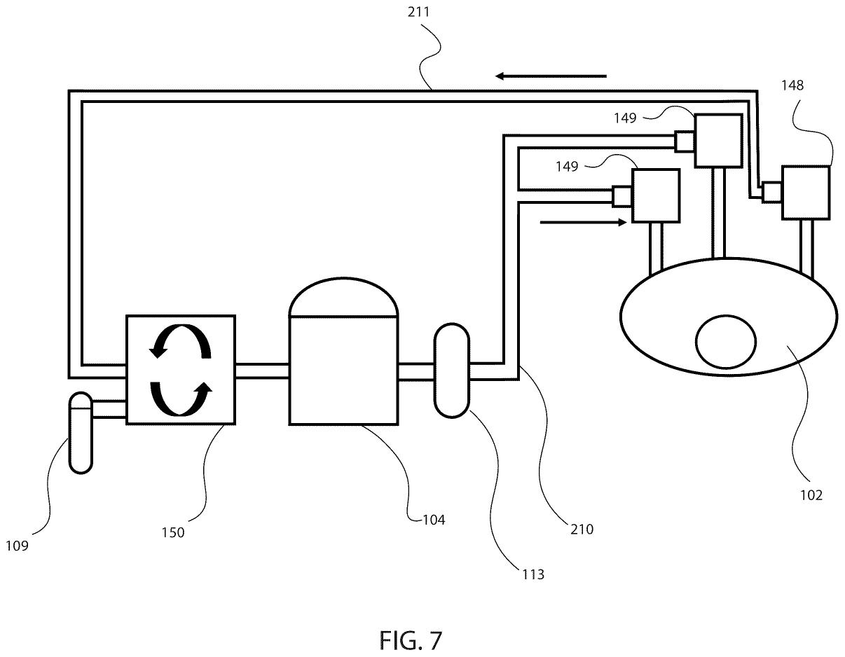

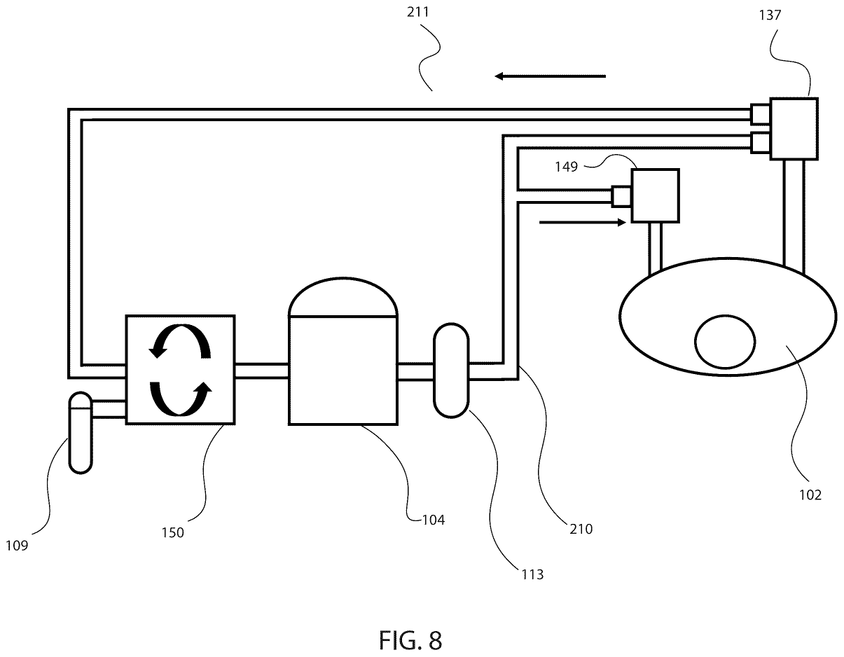

In some configurations, an additional outlet from the surgical cavity may be provided in the systems of FIGS. 7-10. The outlet may be provided by an additional cannula. Where there are multiple outlets from the surgical cavity (for example as shown in FIG. 9) one outlet connected to the recirculation return conduit 211 may instead be used as an outlet. The outlet may be connected to an exhaust line (for example exhaust line 139 as shown in FIG. 6). In some examples, the exhaust line is connected to a suction source (for example suction source 141 as shown in FIG. 6). In some examples, the exhaust cannula and/or line may be connected to a filter to filter vented gases. In some examples, the exhaust line may be provided in in the inlet cannula 149 and/or outlet cannula 138, where the inlet cannula 149 and/or outlet cannula 138 are dual lumen cannulas (for example cannula 137). One or both of the inlet cannula 149 and/or outlet cannula 138 may then be connected to an exhaust line.

The recirculation supply conduit 210 and/or the recirculation return conduit 211 comprise a gas flow pathway.

The recirculation supply conduit 210 and/or the recirculation return conduit 211 may have any of the features of the inlet conduit, or patient conduit as described above.

The system may comprise a condensate management system. The condensate management system may be the condensate management system as described in PCT/NZ2015/050059 which is incorporated by reference in its entirety. A number of condensate management systems are described below, it will be appreciated that each condensate management system may be combined with any combination of other condensate management systems.

The one or more condensate management systems may comprise one or more heaters.

The recirculation supply conduit 210 may comprise a heater.

Additionally, or alternatively, the recirculation return conduit 211 may comprise a heater.

The heater(s) in the recirculation supply conduit 210 and/or the recirculation return conduit 211 may help to prevent condensation of water from the gases as they pass through. For example, the heater(s) may help to keep the temperature of the gases at or above the dew point of the gases. This may be of particular importance when higher dew points are being provided as without the heater(s), water vapour in the gases may condense out of the gases into liquid water in the circuit, in some cases (which may decrease the dew point of the gases provided to the patient, and/or increase the amount of humidity that the humidifier 104 needs to provide to the gases.)

The heater(s) in the recirculation supply conduit 210 and/or the recirculation return conduit 211 may also help the humidifier to maintain an accurate dew point output, and accurate gases temperature output.

The heater(s) in the recirculation supply conduit 210 and/or the recirculation return conduit 211 may also help maintain water vapour in the vapour state, this means that less water needs to be re-evaporated, and it may protect the flow generator from liquid water (as some flow generators may have issues with liquid water).

The heater may be a heater wire.

The heater wire may be located: in a passageway of the conduit, or attached to a wall of the conduit, or embedded in a wall of the conduit.

In some examples, the heater may form part of the wall of the conduit.

The condensate management system may comprise insulation of one or more conduit. For example, the recirculation supply conduit 210 and/or the recirculation return conduit 211 may be thermally insulated. The recirculation supply conduit 210 and/or the recirculation return conduit 211 may comprise thermal insulation. The insulation may be the wall of the conduit itself and/or an additional material added to the external surface of the conduit. The insulation may help to prevent thermal energy leaving the conduit to the environment, cooling down the gases flowing in the conduit and potentially causing condensation.

The condensate management system may comprise a permeable (for example vapour permeable) and/or foamed material. The permeable and/or foamed material may be part of a conduit, or be the wall of a conduit.

The permeable material may be a porous or non-porous material. In some examples the permeable material may be a breathable material.

Water molecules on a first side of the breathable material may be absorbed by the breathable material, diffused through the breathable material, and desorbed to ambient air according to a gradient moving from a higher humidity side to a lower humidity side. This is known as the solution-diffusion mechanism. The solution-diffusion mechanism causes the breathable material to absorb water molecules from the gas and desorb the water molecules to ambient according to a driving gradient moving from the higher humidity side to the lower humidity side.

‘Breathable material’ refers to a non-porous permeable material that allows the passage of water molecules through a monolithic wall of the permeable material via the solution-diffusion mechanism, without allowing the bulk passage of liquid water or bulk flow of respiratory gases all the way through the wall. The rate of transfer is often referred to as a moisture vapor transmission rate (MVTR) or the like).

The breathable material may be for example a sulfonated tetrafluoroethylene-perfluoro copolymer (e.g., Nafion®, a registered mark of E. I. du Pont de Nemours and Company) or a poly(ether-ester) block copolymer (for example, Sympatex®, a registered mark of Sympatex Technologies GmbH), for example but without limitation. In some such configurations, the breathable material can be formed as a film or membrane. In some embodiments the breathable material can be foamed.

When a foamed material is used to form the wall of the conduit, it may also provide additional insulation qualities.

The condensate management system may comprise a fluid trap (for example as a water trap).

The condensate management system may comprise a condenser. In some configurations, the condenser may be part of the water trap.

The condenser can use a less-heated, unheated or cooled material that causes vapour to condense into the condenser. In some configurations, the material can comprise a metal mesh. The material can form a wall or a portion of a wall of the conduit (for example the recirculation supply conduit 210 and/or the recirculation return conduit 211). The material can extend into the lumen of the conduit. Any other suitable material and/or configuration also can be used.

The water trap can comprise a removable and/or reusable chamber. In such configurations, the water trap chamber can be removed, emptied, cleaned and reinstalled. In some such configurations, the water trap chamber can be autoclaved or sterilized between subsequent uses. In some configurations, the water trap does not have a removable chamber; instead, the water trap itself can be removed, emptied, cleaned and reinstalled.

In some configurations the condensate management system may comprise a canister. A desiccant or absorbent material can be contained within the canister. The canister may be connected to one or more conduits in the system.

In some configurations the condensate management system may comprise a desiccant located in the recirculation circuit.

In some configurations the condensate management system may comprise an absorbent material located in the recirculation circuit. In some configurations, the absorbent material may be located in the wall of the conduit.

In some configurations, the condensate management system may allow for the release of trapped condensate back into the gases flow (for example by evaporation). For example liquid in the water trap, desiccant or absorbent material may reevaporate into the gases flow as the gases pass through or over the water trap, desiccant or absorbent material

Where the condensate management system is not part of the conduit itself, the condensate management system may be positioned in the recirculation circuit. The condensate management system may be positioned upstream of the filter 113 and/or between the humidifier 104 and filter 113.

At least one filter (as described above in more detail) may be provided in the gases recirculation circuit.

As shown in FIGS. 2 to 6, a filter 113 may be provided downstream of the humidifier. In some examples, additionally or alternatively, a filter may be provided upstream of the recirculation flow generator 150 (for example filter 111 as shown in FIG. 1 and FIG. 2).

In some examples, a filter is provided between the recirculation flow generator 150 and the humidifier 104.

The system 100 may also comprise a circuit vent path 180. The circuit vent path 180 may provide for a flow path to atmosphere, or a suction source for the system to vent gases. The circuit vent path 180 may assist in purging the system during the start of the procedure and/or to vent gases during operation of the system. For example, in the event that ambient air is entrained into the system, a controlled leak or vent via the circuit vent path 180 may vent gas which includes a combination of therapy gas, e.g. medical CO₂, and entrained ambient air. As therapy gas is input into the system, for example to maintain volume and/or pressure of the surgical cavity, ambient air within the system can be diluted to the extent that it has little or no effect on the system or delivery of intended therapy.

In FIG. 2 the circuit vent path 180 is positioned between the recirculation flow generator 150 and the humidifier 104. In some examples, the circuit vent path may be located downstream of the recirculation flow generator 150 between the recirculation flow generator 150 and the surgical cavity. The circuit vent path 180 could also be incorporated into any of the disclosed systems, such as shown in FIG. 1, and 3-6.

The circuit vent path 180 may comprise an orifice in communication with atmosphere. In some examples the circuit vent path 180 may comprise a valve configured to be controlled to open to vent gases and close to prevent venting of gases by a controller of the system. The circuit vent path 180 may be configured to vent gases at a predetermined flow rate. The predetermined flow rate may be about 2 litres/minute to about 6 litres/minute, or about 2 litres/minute, or about 3 litres/minute, or about 4 litres/minute, or about 5 litres/minute, or about 6 litres/minute.

The circuit vent path 180 may also comprise a filter 181 to filter vented gases. The filter may have the features of the filters as described elsewhere in the specification.

In the system of FIG. 2 a primary flow generator 108 is provided. The primary flow generator 108 is configured to control a volume and/or a pressure of the surgical cavity. The primary flow generator 108 may be that as described with respect to FIG. 1. In some examples, the primary flow generator 108 and flow generator 150 may be a single apparatus i.e. in a single housing.

The primary flow generator and secondary flow generator may communicate with each other (for example via communication between controllers of the primary flow generator and secondary flow generator) to together maintain volume and/or a pressure of the surgical cavity where gases may be lost via leak and/or CO₂ absorption.

In some examples both the primary flow generator 108 and the flow generator 108′ control the volume and/or pressure of the surgical cavity, the primary flow generator 108 controls a primary flow of gases into the surgical cavity, and the flow generator 150 controls a flow rate of the recirculated gases.

A potential advantage of systems which have a primary flow generator 108 and a recirculation flow generator 150 is that as the recirculation flow generator 150 is recirculating gases, it does not significantly impact the operation of the primary flow generator 108. Another potential benefit of this system is that as the recirculated gases do not change the overall volume or pressure at any point in time (as any gas that is removed is reintroduced through recirculation), the primary flow generator 180 can still control of pressure and/or volume of the surgical cavity by the primary flow generator 180.

The system 100 may also comprise an exhaust cannula 138 configured to provide a gases pathway from the surgical cavity to the ambient environment (or suction source) for example as shown in FIG. 6. In the system as shown in FIG. 2 no exhaust is provided, however an exhaust cannula 138 is shown in FIGS. 6 and 7. The exhaust cannula 138 may allow for venting of the surgical cavity to the ambient environment (or suction source). In some examples, the exhaust cannula 138 may be connected to an exhaust line 139. In some examples, the exhaust line is connected to a suction source 141. In some examples, the exhaust cannula may be connected to a filter to filter vented gases.

In some examples, the exhaust cannula may be configured to allow for venting of gases as a predetermined rate. The predetermined rate may be controlled at least in part by the suction source 141, or one or more valves connected to, or part of, the exhaust cannula 138. The exhaust cannula or exhaust line 139 may have a flow controller connected to control the flow through the exhaust cannula 138. The flow controller may be for example a valve that may be manually controllable or controlled by the controller.

The exhaust line may have a valving arrangement that can be manually or automatically operated to allow for venting of the surgical cavity.

The system may be able to be operated in a purge mode to remove unwanted gases from the surgical cavity. Unwanted gases may be for example entrained ambient air, or air present in the system at start up. In the purge mode the system may initially disable the recirculation flow generator, and the primary flow generator i.e. the insufflator may provide gases (for example carbon dioxide) to the surgical cavity while the surgical cavity vents for example through an exhaust cannula and/or through the circuit vent path 180). After an elapsed time, when a specific volume of gas has been provided, and/or when a concentration of carbon dioxide is above a threshold for example above about 90% or above about 95% or above about 99%. The recirculation flow generator can then be activated. In some examples, the humidifier may be activated before the recirculation flow generator.

In some examples the system may be configured to periodically enter a purge mode, or enter a purge mode when a concentration of carbon dioxide is below a threshold, for example below about 90% or below about 95% or below about 99%.

FIGS. 4 and 5 show systems 100 comprising a recirculation flow generator 150, but in contrast to FIG. 2, the system of FIG. 4 does not have a primary flow generator 108. In the embodiment in FIG. 4, the recirculation flow generator 150 is configured to recirculate gases and control the volume and/or pressure of the surgical cavity, in addition to, or alternatively to controlling the flow rate into the surgical cavity.

In some examples, a primary humidifier may be provided in connection with the primary flow generator 108 as shown in FIGS. 2 and 6. This second humidifier may be in addition to the humidifier 104 present in the recirculation circuit.

FIG. 6 shows a system comprising a primary flow generator 108 and a flow generator 108′ (for example the secondary flow generator). The primary flow generator 108 is configured to control a volume and/or a pressure of the surgical cavity (for example by provision of a primary gases flow). The primary flow generator 108 may be configured to be connected to the patient interface (for example as a primary patient interface 136) via a patient conduit 112 to provide the primary flow of gases to the surgical cavity. Although not shown a humidifier may optionally be provided between the primary flow generator 108 and primary patient interface 136 to humidify the primary flow of gases.

The secondary flow generator 108′ is configured to provide a secondary flow of gases. The secondary flow generator 108′ is connected to a humidifier 104. The secondary flow generator may be configured to be connected to the patient interface (for example as a secondary patient interface 136′) via a delivery conduit 210′ to provide the secondary flow of gases to the surgical cavity.

The primary flow generator 108 may be that as described with respect to FIG. 1.

The primary flow generator 108 may be an insufflator.

The secondary flow generator 108′ may generate a secondary flow of gases at a set flow rate.

As described above a primary humidifier may be configured to heat and humidify the gases to a desired dew point and desired temperature.

Having two flow generators, may allow for the primary flow generator 108 to control the volume and/or pressure of the surgical cavity, while the flow generator 108′ (for example the secondary flow generator) provides a flow of gases to the surgical cavity to maintain and/or regulate the body temperature of the patient.

As described above, the humidifier 104 may be controlled to humidify the gases flow to a desired dew point. The desired dew point may allow for energy to be transferred from the gases to the patient when the gases are provided to the patient cavity.

The dew point of the gases may be for example the temperature to which the gases flow must be cooled to become completely saturated with water vapour.

The humidifier 104 introduces energy into the gases flow in the form of the addition of water vapour, which is transferred from the flow of gases to a patient in the surgical cavity of the patient when condensation of water vapour in the gases flow occurs.

The desired dew point may be greater than about 35 degrees Celsius, or greater than about 35.5 degrees Celsius, or greater than about 37 degrees, or greater than about 39 degrees, or greater than about 40 degrees, or greater than above about 41 degrees, or greater than above about 42 degrees.

In some configurations the desired dew point may be based at least in part, on a measurement of a patient temperature and/or patient core body temperature. The desired dew point may be based on an offset above patient temperature and/or patient core body temperature. The offset may be about 1 degree Celsius to about 5 degrees Celsius, or about 1 degree, or about 2 degrees, or about 3 degrees.

The desired dew point may be about 37 degrees Celsius, or about 39 degrees Celsius, or about 40 degrees Celsius, or about 41 degrees Celsius, or about 42 degrees Celsius, or about 43 degrees Celsius.

The desired dew point may be about 35 degrees Celsius to about 50 degrees Celsius, or about 35 degrees Celsius to about 43 degrees Celsius, or about 39 degrees Celsius to about 41 degrees Celsius, or about 38 degrees Celsius to about 41 degrees Celsius, or about 35 degrees Celsius to about 41 degrees Celsius, or about 40 degrees Celsius. In some examples, the dew point may be about 40 degrees Celsius to about 42 degrees Celsius.

The gases flow may be provided to the surgical cavity at a desired gases temperature at or above the dew point. This may prevent condensation of the water vapour before the gases enter the surgical cavity. As described above, the temperature of the gases flow may also allow for energy to be transferred to the patient.

The gases flow may be provided to the surgical cavity at a desired gases temperature within about 1 degree Celsius, or about 2 degrees Celsius from the desired dew point.

The gases flow may be provided to the surgical cavity at a desired gases temperature about 1 degree Celsius, or about 2 degrees Celsius above the desired dew point.

When the gases enter the surgical cavity at the desired dew point, the gases may be cooled to a temperature below the dew point, which causes condensation of the water vapour. The gases may be cooled, for example, when they come into contact with patient tissue which is a lower temperature than the dew point temperature.

The gases may also cool when flowing through the conduits, however this may be mitigated by for example having heaters and/or insulation as described in more detail above. Other condensate management systems may also help to reduce the effect of condensate caused by the cooling of the gases.

FIGS. 7 to 9 show a system similar to FIGS. 4 and 5, with systems 100 comprising a recirculation flow generator 150, which is configured to recirculate gases to and from the patient. Recirculation flow generator 150 can control flow rate into and out of the surgical cavity. The recirculation flow generator 150 may operate independently from or in conjunction with primary flow generator 108 to control the volume and/or pressure of the surgical cavity.

FIGS. 7 to 9 show various examples with multiple inlet cannula and/or outlet cannula to the surgical cavity. Having multiple inlet cannula may help to evenly disperse the flow of gases provided to the surgical cavity by delivering it through multiple cannulas so as to be delivered at multiple locations or through each cannula at a proportion total flow rate. Delivering the gases flow at multiple locations and/or dividing the total flow to the surgical cavity through multiple cannulas may help to prevent mechanical stress and/or heat stress to the patient tissue. This may be important in cases where the cannula does not have specific features to help to diffuse the gases when they enter the surgical cavity.

Having multiple outlet cannula may help to evenly vent gases from the cavity as the cavity can be vented at multiple locations and through multiple cannula each at a proportion of the total flow rate.

Having multiple outlet cannula may help to reduce gases velocity at the outlet of the cannula, as the total flow is spread out, and therefore the velocity of gases at the outlet of each cannula is also reduced.

Having multiple outlet cannula may be beneficial in cannula which do not have features to diffuse flow of gases as it exits the cannula.

In systems where multiple inlets and/or outlet are provided to the patient cavity, the flow rate of gases to the inlets needs to be equalised to the flow rate of gases from the outlets to prevent overpressurisation or underpressurisation of the surgical cavity. The flow rate may be equalised by the flow generator 150 (for example by controlling the pressure and/or volume of the surgical cavity), and/or by pneumatically balancing the inlet and outlet cannulas-for example by balancing the inlet and outlet resistance to flow, and/or through control of the pneumatic characteristics of the tubing and/or connectors.

In some configurations, the flow rate of the inlet and outlet (for example by one or more flow rate sensors) may be measured and controlled to be equal by the flow generator 150.

In some examples, the flow rate of the inlet and outlet (for example by one or more flow rate sensors) may be measured and controlled to be different by an amount by the flow generator 150.

FIG. 7 shows an example of a system which comprises two inlet cannulas 149 and a single outlet cannula 148. In the example of FIG. 7 each inlet cannula provides a proportion of the total flow into the surgical cavity, while the flow out of the surgical cavity is via the outlet cannula 148.

FIG. 8 shows an example of a system where the inlet gases are provided to the cavity through an inlet cannula 149 and a dual lumen cannula for example recirculation cannula 137 (which acts as both an inlet cannula to provide a pathway for gases to the surgical cavity and an outlet cannula to provide a pathway for gases from the surgical cavity).

FIG. 9 shows an example of a system which comprises two dual lumen cannulas for example recirculation cannulas 137. Each recirculation cannula 137 acts as both an inlet cannula to provide a pathway for gases to the surgical cavity and an outlet cannula to provide a pathway for gases from the surgical cavity.

FIG. 10 shows an example of a system which comprises two inlet cannulas 149 and two outlet cannulas 148.

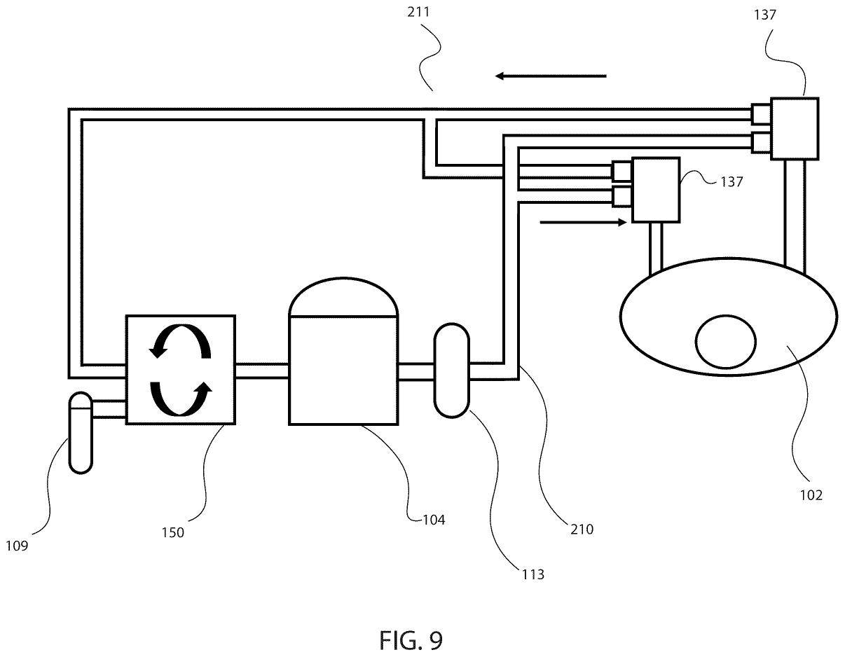

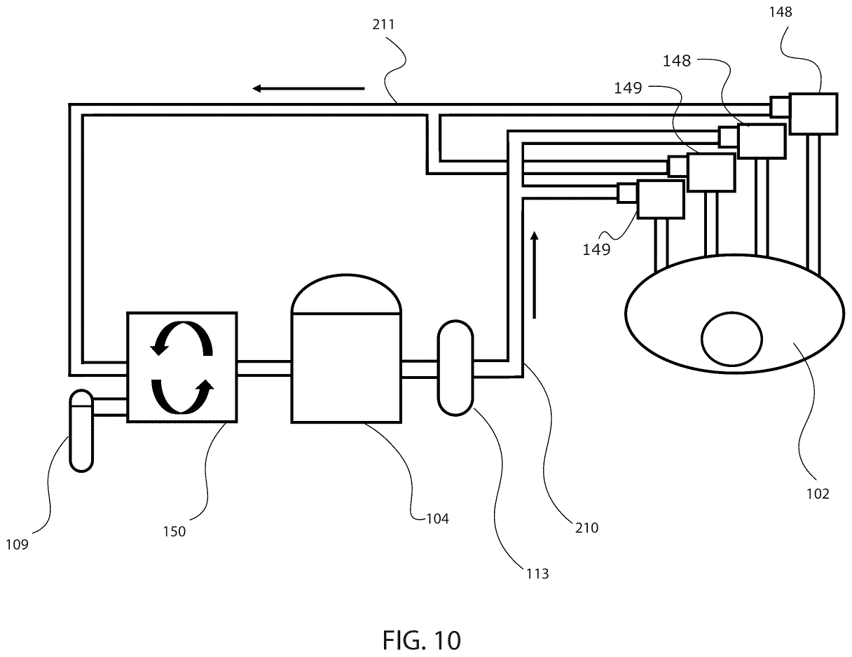

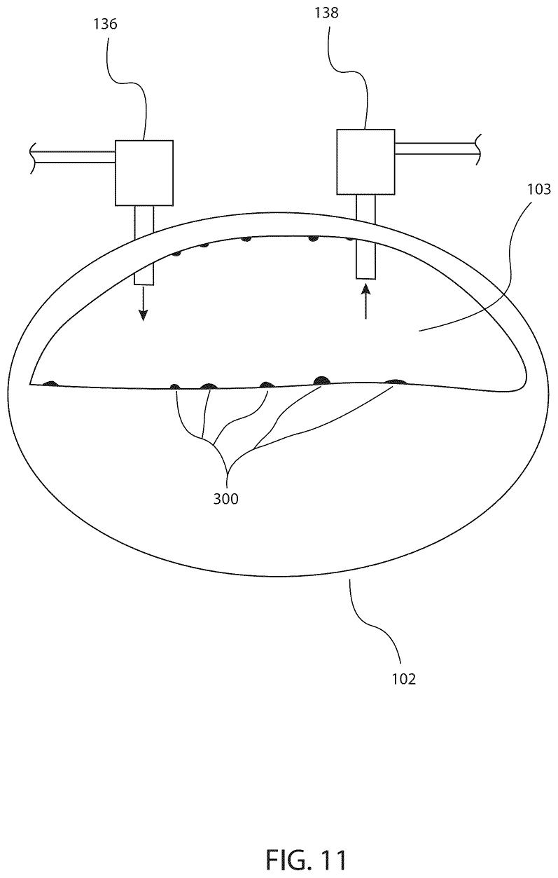

As shown in FIG. 11, the condensation of the water vapour forms on a surface of the surgical cavity 103 as condensate 300. Condensation of the water vapour releases latent heat which warms the tissue of the patient surrounding the surgical cavity.

Condensation may occur on the patient tissue to directly transfer energy to the patient tissue.

Condensation may also occur within the surgical cavity but not on the patient tissue. Heat released from condensation may also be transferred to the patient tissue in this case via the gases.

Further heat transfer may occur between the water vapour in the gases and the tissue of the patient.

The tissue may be for example the patient's organs which are exposed in the surgical cavity.

Blood flow through the tissue which may absorb the heat from condensation may help with heat transfer to the rest of the patient's body. This may help regulate/maintain core body temperature and the surface temperature of the tissue itself (so that it doesn't increase to an unsafe temperature). Blood flow through the tissue may also help to ensure that tissue temperature does not decrease below homeostatic temperatures.

The dew point may be measured (for example by one or more sensors) at or near an outlet of the humidifier, at or near an end of an outlet of the humidifier, at or near an end of a conduit connected to the humidifier, at or near an inlet of a cannula which is configured to deliver the gases flow to the surgical site, at or near an outlet of a cannula which is configured to deliver the gases flow to the surgical site, or in the surgical cavity.

The desired dew point may be at or about atmospheric pressure, or at or about atmospheric+the pressure of the surgical cavity, or about 6 mm HG to about 20 mm HG, or at or about atmospheric+10 mm Hg.

The desired dew point may be a dew point at a location downstream of the humidifier.

The desired dew point may be a dew point at a location: at or near an inlet or outlet of a patient conduit, at or near a patient end or a humidifier end of a patient conduit connected to the humidifier, at or near a beginning of a conduit connected to the humidifier, at or near an inlet of a cannula which is configured to deliver the gases flow to the surgical site, at or near an outlet of a cannula which is configured to deliver the gases flow to the surgical site, or in the surgical cavity.

As described above, the flow generator 108′ (as shown for example in FIGS. 1 and 4) or recirculation flow generator 150 (as shown for example in FIGS. 2 and 5) may control the gases flow to be provided to the surgical cavity 103.

The flow generator 108′ (as shown for example in FIGS. 1 and 6) or recirculation flow generator 150 (as shown for example in FIGS. 2 and 4) may control the gases flow to be provided to the surgical cavity 103 to a desired flow rate.

The desired flow rate may be a flow rate greater than about 3 litres/minute, or greater than about 10 litres/minute, or greater than about 20 litres/minute, or greater than about 30 litres/minute, or greater than about 40 litres/minute, or greater than about 45 litres/minute, or greater than about 50 litres/minute, or greater than about 60 litres/minute.

The desired flow rate may be a flow rate of about 45 litres/minute to about 65 litres/minute. In some examples, the desired flow rate may be about 35 litres/minute to about 60 litres/minute. In some examples, the desired flow rate may be about 45 litres/minute to about 55 litres/minute. In some examples, the desired flow rate may be about 35 litres/minute to about 55 litres/minute. In some examples, the desired flow rate may be about 35 litres/minute to about 60 litres/minute.

The desired flow rate may be a flow rate of about 20 litres/minute, or about 30 litres/minute, or about 40 litres/minute, or about 45 litres/minute, or about 50 litres/minute, or about 55 litres/minute, or about 60 litres/minute, or about 65 litres/minute.

The desired flow rate may be a flow rate of about 40 litres/minute to about 65 litres/minute, or about 45 litres/minute, or about 48 litres/minute, or about 50 litres/minute, or about 51 litres/minute, or about 53 litres/minute, or about 55 litres/minute or about 58 litres/minute, or about 60 litres/minute, or about 62 litres/minute.

The desired flow rate may be a flow rate of about 45 litres/minute to about 65 litres/minute, or about 48 litres/minute, or about 50 litres/minute, or about 52 litres/minute or about 55 litres/minute or about 58 litres/minute, or about 60 litres/minute, or about 62 litres/minute.

The desired flow rate may be a flow rate of about 3 litres/minute to about 120 litres/minute, or about 10 litres/minute, or about 20 litres/minute, or about 40 litres/minute or about 55 litres/minute or about 60 litres/minute, or about 80 litres/minute, or about 100 litres/minute.

It will be appreciated that the greater the flow rate the less energy that needs to be provided from the gases flow per volume of gas. For example, to transfer the same energy to a patient, gases provided at a flow rate of 30 LPM would need to have a higher dew point than gases for example provided at 60 LPM.

Therefore, it will be appreciated that for higher flow rates for example a flow rate flow rate of about 45 litres/minute to about 55 litres/minute, or about 65 litres/minute, the desired dew point may be about 35 degrees Celsius to about 41 degrees Celsius, or greater than about 37, or greater than about 39 degrees Celsius, or about 41 degrees Celsius or about 40 degrees Celsius. In some examples, the desired dew point may be about 42 degrees Celsius or about 43 degrees Celsius.

The desired flow rate may be a continuous flow rate.

It will be appreciated that the continuous flow means that a positive amount of gas flow is delivered into the body cavity. The positive gas flow may be steady (a flat or constant flow rate), pulsatile, or irregularly continuous (random).

Providing the gases flow at the flow rates as described may allow for more energy to be transferred to the patient. However, the flow at which the gases are provided needs to be balanced with consideration of the dwell time of the gases in the surgical cavity. If the flow rate is too large then the gases may not be able to transfer any additional heat to the patient compared to lower flow rates as they are moved through the surgical cavity too quickly. In other words, a flow rate limit may exist where no, or little heat transfer occurs when flow rate is increased above the limit (assuming other characteristics of the gases flow remain constant).

As described above the system 100 may control the temperature of the gases to a desired temperature.

In some examples, the gases may be heated (optionally, in addition to humidifying) by the humidifier and/or a heater of a connected conduit (for example a patient conduit as shown in FIGS. 1 and 6, or a recirculation conduit 210 as shown in FIGS. 2 and 4).

The greater the temperature of the gases the greater the amount of energy the gases can contain and therefore the greater energy that can be transferred to the patient tissue surrounding the surgical cavity. However, gases temperatures which are too high may cause damage to patient tissue.

The desired gases temperature may be greater than the desired dew point of the gases flow.

The desired gases temperature may be an offset plus the desired dew point of the gases flow. In some examples the offset may be about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius).

In some examples the desired gases temperature may be a based on the desired dew point for example the desired gases temperature may be a function of the desired dew point.

The desired gases temperature may be about 38 degrees Celsius to about 43 degrees Celsius, or about 40 degrees Celsius to about 43 degrees Celsius, or about 40 degrees Celsius to about 45 degrees Celsius, or about 40 degrees Celsius to about 45 degrees Celsius.

The desired gases temperature may be a within a range about the desired dew point temperature (for example within about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius, or about 1 degrees Celsius, or about 2 degrees Celsius of the desired dew point).

As described above, the desired temperature may be a temperature at or near an outlet of a cannula which is configured to deliver the gases flow to the surgical site. In some examples, the desired gases temperature may be a dew point at or near an inlet or outlet of a patient conduit 112, at or near a patient end or a humidifier end of a patient conduit 112 connected to the humidifier, at or near a beginning of a conduit connected to the humidifier, or in the surgical cavity.

The gases flow may be provided at a flow rate, humidity and/or temperature so as to reach or maintain normothermia for a patient. Normothermia may be defined as a normal patient core body temperature, for example between about 36 and 38 degrees Celsius.

A patient may be considered to be hypothermic for example if the patient core body temperature is below 35 degrees Celsius.

The system may be configured to control at least one gases parameter of the gases flow to lower heat transfer to the patient when patient heating is not required, and to control the at least one gases parameter of the gases flow to increase heat transfer to the patient when patient heating is required.

The system may be configured to control at least one gases parameter based on one or more patient heating criterion. The patient heating criterion may be indicative of whether the patient does or does not require heat to be transferred to the patient from the system. The patient heating criterion may be based on input from a user and/or one or more sensor measurements for example a patient temperature sensor. For example, if the patient heating criterion is not satisfied, then the system may be configured to control at least one gases parameter to at least one first desired gases parameter, and if the patient heating criterion is satisfied then the system may be configured to control at least one gases parameter to at least one second desired gases parameter.

In some examples, the system may be configured to control at least one gases parameter based on whether a patient does or does not require heating.

In some examples, the system may be configured to control at least one gases parameter based on how much heating is required.

It will be appreciated that even if a patient does not require heating, energy may still be transferred from the gases flow to the patient for example.

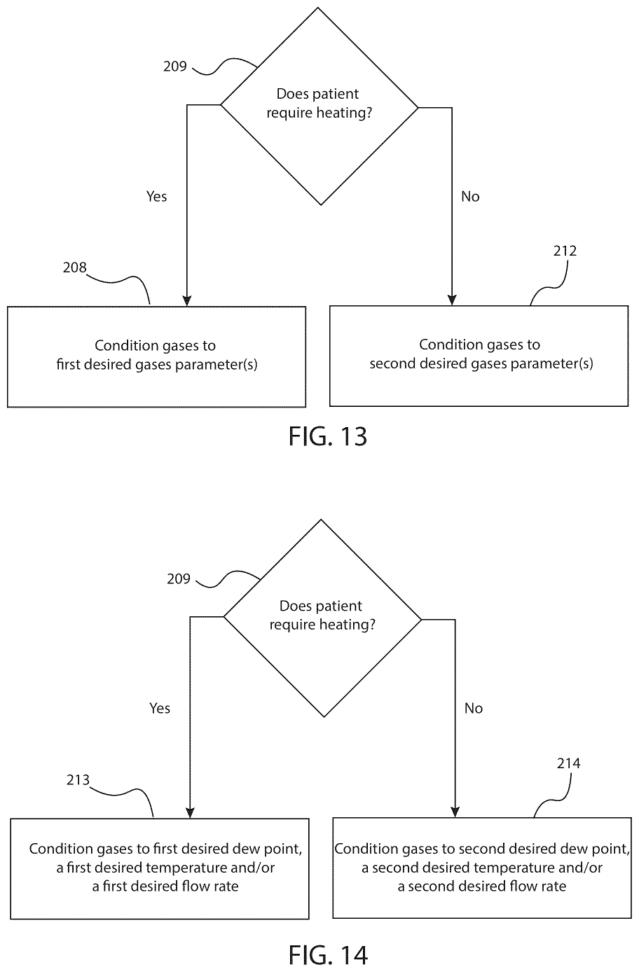

FIG. 13 shows an example of control of the at least one gases parameter based on whether a patient requires heating. As shown for example in FIG. 13, at step 209 it is determined if the patient requires heating. If the answer is yes and the patient does require heating the at least one gases parameter may be controlled to at least one first desired gases parameter as shown at step 208. If the answer is no, and the patient does not require heating the at least one gases parameter may be controlled to at least one second desired gases parameter as shown at step 212.

The at least one second desired gases parameter may be lower than the than the corresponding at least one first desired gases parameter. Lowering the at least one gases parameter may reduce the heat transfer from the gases to the patient.

In some examples, the second desired dew point is lower than the first desired dew point.

In some examples, the second desired flow rate is lower than the first desired flow rate.

In some examples, the second desired temperature is lower than the first desired temperature.

The at least one gases parameter may comprise any combination of a dew point of the gases flow, a temperature of the gases flow, or a flow rate of the gases flow.

The at least one first desired gases parameter may be any combination of a first desired flow rate and/or a first desired temperature and/or a first desired dew point.

In some examples, the first desired flow rate is about 20 litres/minute, or about 30 litres/minute, or about 40 litres/minute to about 55 litres/minute, or about 65 litres/minute. In some examples, the first desired flow rate may be the flow rate as described above.

In some examples, the first desired dew point is about 35 degrees Celsius to about 43 degrees Celsius, or about 38 degrees Celsius to about 41 degrees Celsius, or about 40 degrees Celsius. In some examples, the first desired dew point may be the dew point as described above.

In some examples, the first desired temperature is about equal to the first desired dew point, above the first desired dew point, about an offset plus the first desired dew point, and/or based on the first desired dew point. The offset may be about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius. In some examples, the first desired temperature may be the temperature as described above.

In some examples, the first desired temperature is about 38 degrees Celsius to about 43 degrees Celsius.

The at least one second desired gases parameter may be any combination of a second desired flow rate and/or a second desired temperature and/or a second desired dew point.

In some examples, the second desired flow rate is about 10 litres/minute to about 30 litres/minute, or about 10 litres/minute to about 40 litres/minute, or about 10 litres/minute.

In some examples, the second desired flow rate is below about 10 litres/minute, or below about 20 litres/minute, or below about 30 litres/minute, or below about 40 litres/minute.

In some examples, the second desired flow rate is above a predetermined threshold.

In some examples, the predetermined threshold is 6 litres/minute, or about 10 litres/minute.

In some examples, the second desired dew point is about 33 degrees Celsius to about 37 degrees Celsius, or about 34 degrees Celsius, or about 37 degrees Celsius.

In some examples, the second desired temperature is about equal to the second desired dew point, above the second desired dew point, about an offset plus the second desired dew point, and/or based on the second desired dew point.

In some examples, the second desired gases temperature is 37 degrees Celsius to about 43 degrees Celsius, equal to the desired dew point, or above the desired dew point.

It will be appreciated that in some configurations some of the at least one first and second desired gases parameters are the same (i.e. at least one gases parameter does not change based on whether the patient does or does not require heating).

FIG. 14 shows an example of control of the at least one gases parameter, where the at least one gases parameter are dew point, temperature and/or flow rate of the gases, based on whether a patient requires heating.

As shown for example in FIG. 14, at step 209 it is determined if the patient requires heating. If the answer is yes and the patient does require heating the dew point, temperature and/or flow rate of the gases may be controlled to a first desired dew point, a first desired temperature, and/or a first desired flow rate as shown at step 208. If the answer is no, and the patient does not require heating the dew point, temperature and/or flow rate of the gases may be controlled to a second desired dew point, a second desired temperature, and/or a second desired flow rate as shown at step 212.

The system may automatically (for example via one or more sensor measurements), and/or manually (for example via input from a clinician) determine whether a patient does or does not require heating.

In some configurations, the system may determine that a patient does, or does not require heating based on an input from a user such as a clinician. The input may be provided by the user via a user interface. The user interface may for example be provided on the humidifier and/or flow generator and/or a separate device which is part of the system.

In some configurations, the system may determine that a patient does, or does not require heating based on one or more measure patient parameters. The patient parameters may be measured by one or more sensors.

In some configurations, the system may determine that a patient does, or does not require heating based on an output of one or more sensors (for example as a patient heating criterion). The one or more sensors may be a patient temperature sensor. The one or more patient temperature sensors may be configured to measure an external patient temperature and/or patient core body temperature. The one or more patient temperature sensors may provide a sensor output to the system which is indicative of an external temperature of a patient and a patient core body temperature.

In some examples, the system may control at least one gas parameter based on an external patient temperature and/or patient core body temperature. For example, one or more of the dew point of the gases flow, temperature of the gases flow, and/or flow rate of the gases flow may be based on the external patient temperature and/or patient core body temperature. In some examples, the dew point of the gases flow, temperature of the gases flow, and/or flow rate of the gases flow is negatively related to the external patient temperature and/or patient core body temperature. For example, if the patient temperature is lower, this may indicate more heat transfer is required and so one or more of the dew point of the gases flow, temperature of the gases flow, and/or flow rate of the gases flow may be increased to increase heat transfer. Conversely if the patient temperature is higher, this may indicate less heat transfer is required and so one or more of the dew point of the gases flow, temperature of the gases flow, and/or flow rate of the gases flow may be decreased to decrease heat transfer.

In some examples, a clinician may indicate the degree of heat transfer required for example via a user interface. If the clinician indicates more heat transfer is required, one or more of the dew point of the gases flow, temperature of the gases flow, and/or flow rate of the gases flow may be increased to increase heat transfer. Conversely, if the clinician indicated less heat transfer is required, one or more of the dew point of the gases flow, temperature of the gases flow, and/or flow rate of the gases flow may be decreased to decrease heat transfer.

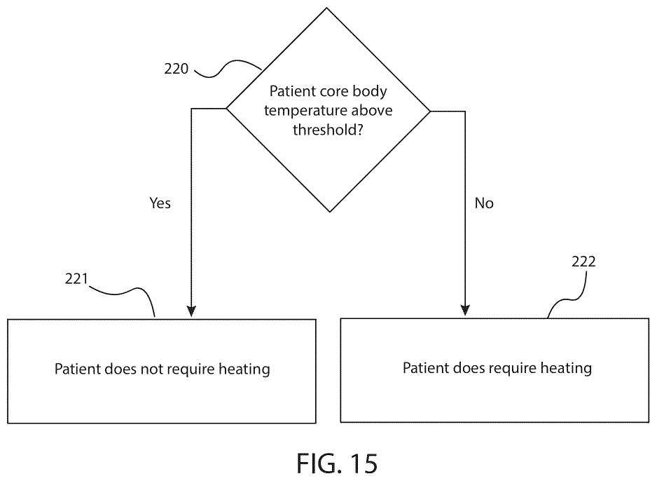

FIG. 15 shows an example of determining whether a patient requires heating, or does not require heating. As shown in FIG. 15, if a patient's core body temperature, and/or external temperature is above a threshold (for example a patient heating criterion is satisfied) at step 220, it is determined that a patient does not need heating at step 221. Further, if the patient's core body temperature and/or external temperature is below a threshold (for example a patient heating criterion is not satisfied) at step 220 it is determined that a patient does need heating at step 222.

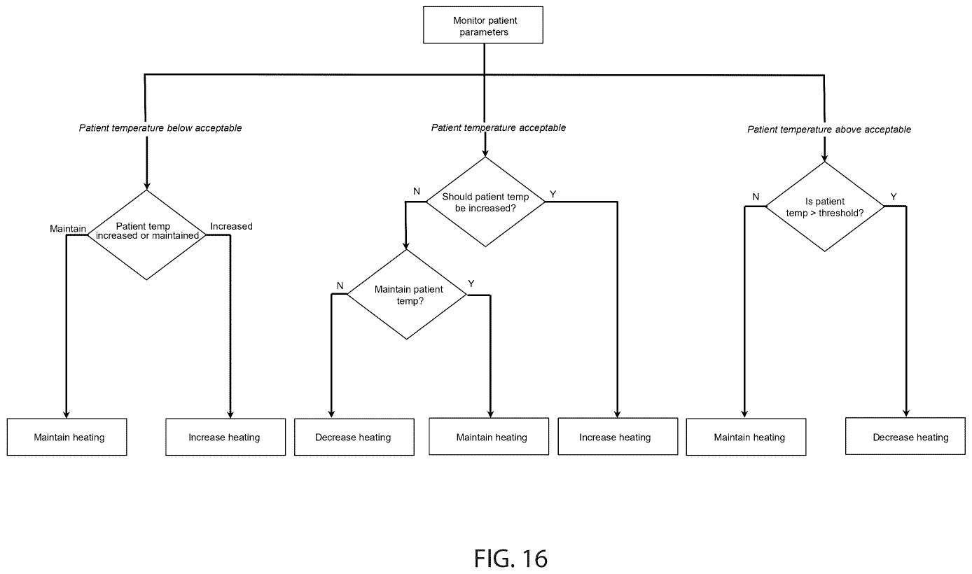

FIG. 16 shows a flow chart outlining an example of control of the system and/or a method of providing gases to a patient undergoing a surgical procedure. As described above, the system may be controlled based on input from a clinician (for example an anaesthetist), and/or based on the one or more patient parameters (for example as measured by one or more sensors.) The control may be undertaken by a clinician, and/or by one or more controllers of the system.

The flow chart in FIG. 16, shows three options for categorizing the patient based on the one or more patient parameters comprising at least patient temperature. The patient may be categorised into the patient temperature below acceptable, patient temperature acceptable or patient temperature above acceptable. The controller of the system may categorise the patient, or the categorisation may be made by the clinician. The categorisation may be based on an external patient temperature and/or patient core body temperature compared to a threshold or range for each category.

For avoidance of doubt patient temperature in FIG. 16 may be for example external patient temperature and/or patient core body temperature.

In some configurations, the categorisation may be made based on a rate of change of a patient parameter (for example a rate of change of a temperature). The rate of change may be compared to an associated threshold.

The clinician may select a category based on information provided by the system (for example patient temperature and/or operating parameters), and/or other information available on the patient which may be from other sensors or systems external to the system (i.e. other patient monitoring). The clinician may then control one or more operating parameters of the system either directly, or for example by instructing the system to increase, decrease or maintain the heating to the patient.

In some examples. the system may select a category and control components of the system based on which category is selected.

The below disclosure is directed towards the clinician making decisions on categorisation and operation of the system, however it will be appreciated the logic may be implemented by the system (via for example one or more controllers).

The clinician may select the patient temperature below acceptable category when the patient temperature is below a below acceptable threshold, or within a below acceptable range. In some configurations the clinician may select the patient temperature below acceptable category when the rate of change of the patient temperature is less or more than a below acceptable rate of change threshold. For example, the threshold may be a decrease in temperature of a time period.

The clinician may select the patient temperature acceptable category when the patient temperature is within an acceptable patient temperature range. The acceptable patient temperature range may be for example a temperature range, or a rate of temperature change range. The acceptable temperature range may be a normothermic range for example between about 36 and 38 degrees Celsius, however what is an acceptable temperature range may vary between clinician and based on other factors as described in more detail below).

The clinician may select the patient temperature above acceptable category, when the patient temperature exceeds an above acceptable threshold. The above acceptable patient temperature threshold may be for example a temperature range, or a rate of temperature change range. For example, if the rate of temperature change exceeds the above acceptable threshold then the patient may be categorised as being above acceptable.

The thresholds associated with each category may change based on one or more operating parameters of the system and/or the mode the system is operating in and the type of cannula connected.

The categorisation and/or one or more of the operating parameters of the system (for example desired dew point, desired temperature and/or desired flow rate of the flow of gases to the patient cavity) may be based at least in part on other factors such as respiratory information (for example respiratory rate), cardiovascular information (for example heart rate and/or blood pressure), the type of procedure, the expected length of the procedure, the patient condition (for example comorbidities), patient size (for example BMI), patient body fat percentage, and/or ambient temperature (for example as an environmental factor).

The clinician may enter these factors into the system via the user interface, and the system may change one or more thresholds associated with each category accordingly.

In some configurations, the clinician themselves may take these factors into account when setting various thresholds and/or determining operating parameters of the system.

In one example, if the ambient temperature is low and/or the patient is low body fat then the decision may be made to increase patient temperature to prevent cooling, as the clinician may anticipate that patient temperature to drop over the course of the procedure although the current patient temperature is acceptable.

The system may vary one or more thresholds based on input from a clinician. In some configurations, the clinician may determine the thresholds.

The clinician may directly change the operating parameters of the system to increase, decrease or maintain heating, and/or may provide an input (i.e. to increase, decrease or maintain heating), to the system. The system may control the operating parameters. Heating in this context may be a function of one or more of the operating parameters of the system for example dew point, gases temperature and flow rate. It will be appreciated that a change in heating may not necessarily cause a corresponding change in patient temperature, as patient temperature may be affected by other factors (for example a decreasing ambient temperature, effect of anaesthetic drugs, and the like).

The system may be operated to provide heating to the patient. One or more of the dew point of the gases flow, temperature of the gases flow, and/or flow rate of the gases flow may be initially set. For example, these may be set by the system and/or with reference to clinician preferences or requirements.

One or more of the dew point of the gases flow, temperature of the gases flow and/or flow rate of the gases flow may be set such that gas flow delivered to the surgical cavity compensates for or overcomes evaporative cooling effects of gas flow in the surgical cavity.

One or more of the dew point of the gases flow, temperature of the gases flow and/or flow rate of the gases flow may be set so as to take into account smoke clearance in the system.

The heating provided by the system may allow for heat transfer from the gases to the patient for example via one or more of the mechanisms as described elsewhere in the specification. Changing the heating provided by the system may change the amount of energy available in the gases for heat transfer to the patient.

The system may be operated to maintain heating provided to the patient. When heating is maintained, one or more of the dew point of the gases flow, temperature of the gases flow, and/or flow rate of the gases flow may be maintained.

The system may be operated such that the gas flow parameters overcome or at least partly compensate for evaporative cooling effects from gas flow in and/or through the surgical cavity.

The system may be operated to increase heating. When heating is increased, one or more of the dew point of the gases flow, temperature of the gases flow, and/or flow rate of the gases flow may be increased.

Operation of the system to increase heating may increase heat transfer to the patient in use.

The system may be operated to decrease heating. When heating is decreased, one or more of the dew point of the gases flow, temperature of the gases flow, and/or flow rate of the gases flow may be decreased. In some examples, when heating is decreased the system may deactivate the humidifier. However, it will be appreciated that the system may still provide humidified gases to the patient when therapy is off as there may be residual humidity in gases provided to the surgical cavity.

When the system is operated to decrease heating, heat transfer to patient may also be decreased.

It will also be appreciated that in FIG. 16, increasing heating, decreasing heating and/or maintaining heating may instead be replaced with increasing patient temperature, decreasing patient temperature, or maintaining patient temperature. In these cases, the system (or clinician) may control one or more operating parameters to control the patient temperature according to the category for example via closed loop control.

When the clinician observes the patient temperature below acceptable category the clinician may determine whether to increase patient temperature or maintain patient temperature. The clinician may provide input to the system to control whether the patient temperature should be increased or maintained and/or whether heating should be increased or maintained. Additionally, or alternatively, the clinician may determine whether to increase or maintain patient temperature based at least in part on a measured patient temperature. When patient temperature is maintained, the system may maintain heating. When patient temperature is increased the system may increase heating.

When the clinician observes the patient temperature is acceptable category the clinician may first determine whether to increase patient temperature. The clinician may provide to the system to control whether the patient temperature should be increased and/or whether heating should be increased. Additionally, or alternatively, the clinician may determine whether to increase patient temperature based on a measured patient temperature. When patient temperature is increased the system may increase heating. When patient temperature is not increased the system may further determine whether to maintain patient temperature. The clinician may provide input to the system to control whether the patient temperature should be maintained and/or whether heating should be maintained. Additionally, or alternatively, the clinician may determine whether to maintain patient temperature based on a measured patient temperature. When patient temperature is maintained, the system may maintain heating When patient temperature is not maintained the system may decrease heating.

When the clinician observes the patient temperature is above acceptable category the clinician may first determine whether the patient temperature is above an acceptable threshold. When patient temperature is above the threshold the system may decrease heating. When patient temperature is below the threshold, the system may maintain heating.

The system may be configured to operate in one or more modes. The modes may be selected at least partly based on a user interface, based on, at least in part, for example, what surgical access device(s) is used in the system. Mode may be transitioned based at least in part upon input to the system (for example sensor inputs).

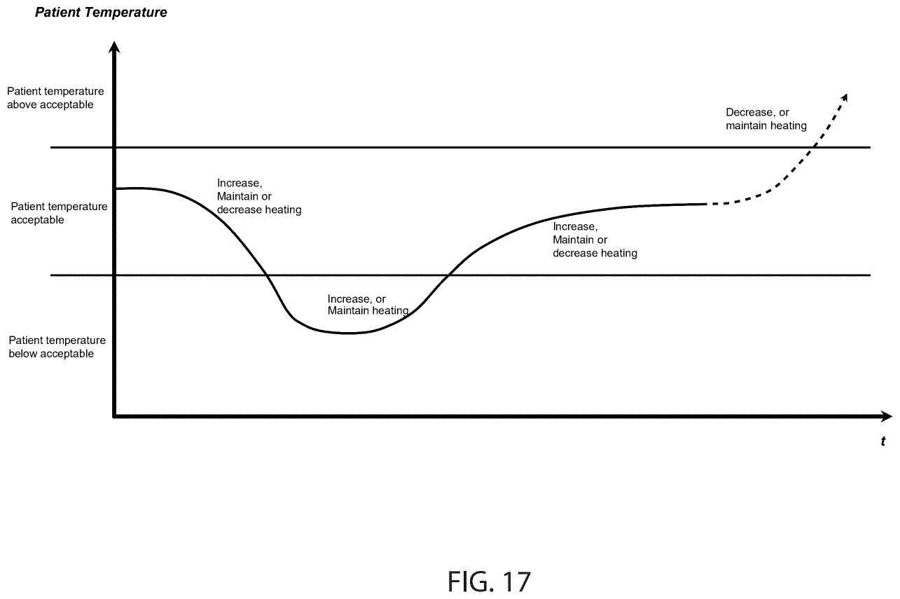

FIG. 17 shows an example patient temperature trajectory (for example external patient temperature and/or patient core body temperature) over time before, during and after a typical minimally invasive surgical procedure. The patient temperature trajectory provides an indication of how a patient temperature generally trends into patient temperature categories of: patient temperature below acceptable patient temperature acceptable, and patient temperature above acceptable.

The patient temperature categories as shown in FIG. 17 are illustrative, and as described above the patient temperature categories may vary based on a number of factors.

FIG. 17 shows that when the patient temperature is below acceptable, heating and/or patient temperature may be increased or maintained. Further, if patient temperature is below acceptable then heating and/or patient temperature may be increased, maintained or decreased. If patient temperature is above acceptable (for example as shown with the dashed part of the line) then heating and/or patient temperature may be maintained or decreased.

The system may identify the cannula connected to the system and based on the identified cannula operate the system according to a mode associated with the identified cannula. The mode associated with the identified cannula may define one or more operating parameters, and/or operating parameter limits.

The system may identify the cannula connected to the system and if the cannula is not identified, the system may instead operate in the default mode.

The one or more modes may have associated programs which are executed by one or more controllers of the system.

Each mode may be associated with a single identified cannula or a group of identified cannulas.

The one or more operating parameters, and/or operating parameter limits defined by the one or more modes, may be based on, at least in part, characteristics of the identified cannula. The characteristics may be one or more physical characteristics (for example the presence of diffusion apertures in the cannula), and/or one or more operational characteristics (for example a maximum allowed flow rate, maximum allowed dew point, maximum allowed gases temperature of and/or resistance to flow of the cannula).

The operating parameters defined by each mode may be at least in part, based on the characteristics of the identified cannula. For example, if a cannula does not have adequate physical and/or operational characteristics for safe delivery of gases at a particular dew point, gases temperature, and/or mode flow rate the operating parameters may be reduced, or a limit placed on the operating parameters so that therapy is delivered safely.

In some configurations, if the identified cannula does not have physical characteristics, such as diffusion features (i.e. diffusion apertures 1350), which may assist to prevent thermal stress and/or mechanical stress on patient tissue when the flow of gases is conditioned according to some operational parameters (for example high flow rate), then the mode associated with that identified cannula may have a lower flow rate (compared to other cannulas) and/or the flow rate may be limited.

In some configurations, if the identified cannula has one or more operational characteristics, the one or more operating parameters, and/or operating parameter limits defined by the one or more modes, may be based on, at least in part, the operational characteristics. For example, if the cannula has a maximum allowable flow rate, then the operating parameter of the flow rate of the gases may be limited to the maximum allowable flow rate.

If the cannula has a maximum allowable dew point, then the operating parameter of the dew point of the gases may be limited to the maximum allowable dew point.

If the cannula has a maximum allowable gases temperature, then the operating parameter of the gases temperature of the gases may be limited to the maximum allowable gases temperature.

During operation of the system, the operating parameters may be limited to the operating parameter limits.

The maximum allowable flow rate, maximum allowable dew point and/or maximum allowable gases temperature may be based on patient safety considerations and patient outcomes for the identified cannula when used with the system.

In some examples, the one or more operational characteristics may comprise operability and risk factor information of the identified cannula. The one or more operating parameters, and/or operating parameter limits may be based on the operability and risk factor information. For example, the risk factor information may comprise the risk of complications during use of the cannula such as the risk of embolism. In some examples, the operability information may be based on condensation associated the cannula.

When a cannula cannot be identified, the system may operate in a default mode, for example deliver the gases to a lower dew point, gases temperature, and/or flow rate of the gases flow relative to the modes for which a cannula can be identified. For example, in the default mode, the system may condition the gases to default mode gases parameters such as a default mode dew point, a default mode gases temperature, and/or a default mode flow rate. The default mode parameters may be based on the corresponding mode parameters for which a cannula can be identified, but with one or more parameters reduced by a set amount, or a set percentage.

In some configurations, in the default mode the default mode gases temperature is the same as the mode for which a cannula can be identified gases temperature, and the default mode for which a cannula can be identified dew point is lower than the mode for which a cannula can be identified dew point and the default mode flow rate is lower than the mode gases flow rate.

In some configurations the default mode dew point may be such that there is net positive energy transfer to patient to actively warm and/or maintain patient temperature.

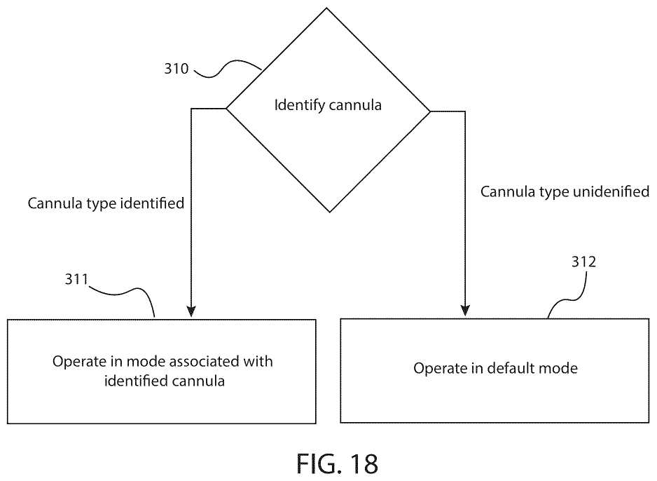

FIG. 18 shows an example of the system operating in a particular mode based on identification of a connected cannula is detected. At step 310 the system identifies a connected cannula. If a cannula is identified then the system may operate in a mode associated with the identified cannula at step 311, if a cannula is not identified then the system may operate in a default mode at step 312.

The method of identifying what cannula(s) are connected may be for example based on a wired or wireless connection between the cannula(s) and one or more controllers of the system. The identification of what cannula is connected may be based on one or more electrical characteristic of the cannula measured by the controller (for example a resistance of an identification resistor) or based on data transferred to the controller, or between the controller and cannula.

The method of identifying what cannula(s) are detected may be based on a flow and/or pressure characteristic of the cannula. For example, the resistance to flow of the cannula may be measured and/or a known flow rate of gases may be provided and the pressure characteristics of the cannula may be measured.

A cannula may be determined to not be identified, when the cannula is not recognised by the system.

The systems as described above may not have issues with smoke (given the high flow rate of gases through the cavity which aids in clearing smoke from the surgical cavity) while still providing gases at desired operating parameters, meaning smoke evacuation can occur without compromising therapy.

In some configurations the system may be configured to operate in a smoke evacuation mode. The system may operate in smoke evacuation mode to remove smoke from the surgical cavity and/or other parts of the system. During the smoke evacuation mode, the system may reduce the output of the humidifier and/or deactivate the humidifier. In some configurations, the humidity may be reduced by reducing the dew point of the gases.

In some configurations, when the system operates in the smoke evacuation mode, the system may be configured to maintain the humidity level (i.e. the dew point of the gases) and/or the temperature of the gases.

In some configurations, when the system operates in the smoke evacuation mode, the system may increase the flow rate of the gases so as to remove smoke from the surgical cavity (to be filtered out by one or more filters in the system (for example in a recirculation system) and/or exhausted through a venting cannula.

When the system operates in the smoke evacuation mode with a recirculation circuit (as described above), then this may reduce heat losses (and reduce humidity losses for example through condensation).

The system may operate in a smoke evacuation mode when the system receives an input from a user via a user interface. The user interface may for example be provided on the humidifier and/or flow generator and/or a separate device which is part of the system.

The smoke evacuation mode may operate until the user turns the smoke evacuation mode off.

The smoke evacuation mode may operate until the system detects no smoke in the system.

Once the system exits smoke evacuation mode the system may return or be set to the mode and/or operating parameters of the system was previously operating in.

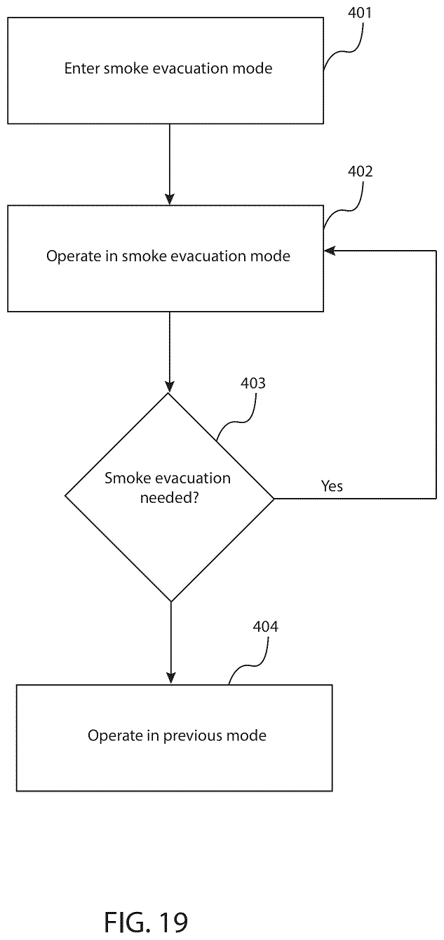

FIG. 19 shows an example of operation of the smoke evacuation mode. At step 401 the system enters the smoke evacuation mode (for example as described above).

At step 402, the system operates in the smoke evacuation mode.

At step 403 the system determines whether smoke evacuation is still needed (for example smoke is still detected or a user input is detected). If smoke evacuation is still needed the system proceeds to step 402 and continues to operate in the smoke evacuation mode.

If smoke evacuation is not needed, the system exits smoke evacuation mode the system returns to the mode the system was previously operating in at step 404.

Also disclosed is a method of transferring heat to a patient undergoing a surgical procedure. As described above the gases flow may be conditioned by controlling parameters of the gases flow (for example one or more of: a dew point, a gases temperature, and/or a flow rate), the gases flow is then delivered to the surgical cavity.

The gases may be conditioned by the system as described above. For example, the gases may be provided by the system as shown in FIGS. 2-6.

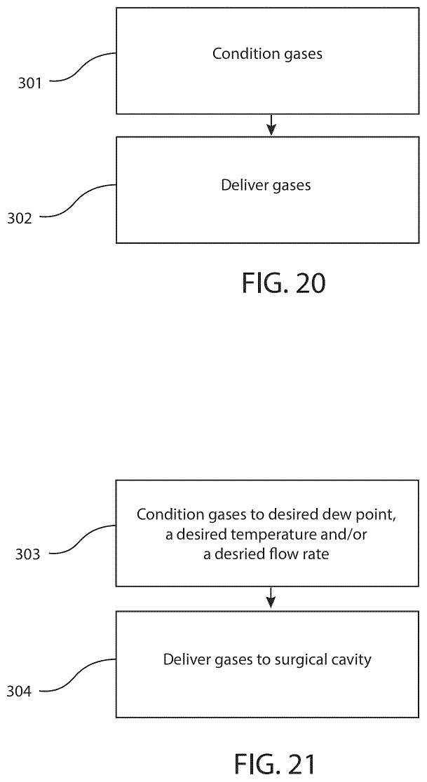

FIG. 20 shows an example of a method. At step 301, the gases are conditioned, and at step 302 the gases are delivered (for example to a conduit).

FIG. 21 shows a further example of a method. At step 303, the gases are conditioned to a desired dew point, a desired temperature, and a desired flow rate, and at step 304 the gases are delivered to the surgical cavity.

The gases may be delivered to a conduit at described above in more detail which is suitable for inclusion (for example insertion) in a cavity of a patient. The conduit may be connected to for example a cannula as described above, and inserted into a cavity of the patient.

In the disclosed method the gases may be conditioned to have a dew point of about 35 degrees Celsius to about 43 degrees Celsius, or about 38 degrees Celsius to about 41 degrees Celsius and a temperature about equal to the desired dew point, above the desired dew point, about an offset plus the desired dew point, and/or based on the desired dew point. It will be appreciated that the dew point and temperatures of the gases as conditioned as part of the method may be as the gases flow described above with respect to the system, apparatus and/or method.

In the disclosed method the gases may be conditioned to have a flow rate of about 3 litres/minute to about 120 litres/minute, or about 45 litres/minute to about 65 litres/minute, or about 20 litres/minute. It will be appreciated that the flow rate at which the gases flow is provided may be as the gases flow described above with respect to the system, apparatus and/or method.

Also disclosed is a method of controlling a surgical humidification system according to one or more operating parameters as disclosed above.

Also disclosed is a composition. The composition is for use in a method of transferring heat to a patient undergoing a surgical procedure. The use of the composition may help to prevent a patient from unintended perioperative hypothermia. The composition is delivered to the surgical cavity (for example as part of the method of transferring heat to a patient undergoing a surgical procedure).

The composition may be prepared by the systems, apparatuses and/or methods as described above.

The composition may be provided as per the gases flow as described above.

The composition may have a dew point of about 35 degrees Celsius to about 41 degrees Celsius, or about 38 degrees Celsius to about 43 degrees Celsius and at a temperature of about 40 degrees Celsius to about 43 degrees, an offset plus the dew point, or above the dew point or above the dew point. It will be appreciated that the dew point and temperatures of the composition may be as the gases flow described above with respect to the system, apparatus and/or method.

The composition may be delivered to the surgical cavity of the patient at a flow rate of about 3 litres/minute to about 120 litres/minute, or about 45 litres/minute to about 65 litres/minute. It will be appreciated that the flow rate at which the composition is provided may be as the gases flow described above with respect to the system, apparatus and/or method.

The composition may comprise carbon dioxide. In some configurations, the composition may be any combination of ambient air, Oxygen, Carbon Dioxide and/or one or more inert gases (e.g. argon, helium and nitrous oxide). As described above, a correction factor may be applied to desired gases parameters depending on the combination of gases in the composition.

Although certain examples are described above, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described below.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication. For example, actions such as “controlling a motor speed” include “instructing controlling of a motor speed.”

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. In some embodiments, the computer system may be a cloud-based computing system whose processing resources are shared by multiple distinct business entities or other users.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A surgical humidification system, comprising:
a flow generator configured to control a gases flow to be provided to a surgical cavity at a desired flow rate, wherein the desired flow rate is a flow rate of about 3 litres/minute to about 120 litres/minute,
a humidifier configured to humidify the gases flow to be provided to the surgical cavity, and
wherein the humidifier is configured to humidify the gases flow to a desired dew point of about 35 degrees Celsius to about 43 degrees Celsius, or about 40 degrees Celsius.

2. A surgical humidification system, comprising:
a flow generator configured to control a gases flow to be provided to a surgical cavity at a desired flow rate, wherein the desired flow rate is a flow rate of about 45 litres/minute to about 65 litres/minute,
a humidifier configured to humidify the gases flow to be provided to the surgical cavity, and
wherein the humidifier is configured to humidify the gases flow to a desired dew point of about 38 degrees Celsius to about 41 degrees Celsius.

3. The surgical humidification system of claim 1 or 2, wherein the humidifier and/or a heater of a patient conduit pneumatically connected to the humidifier, is configured to heat the gases flow to a desired temperature of about 38 degrees Celsius to about 43 degrees Celsius.

4. The surgical humidification system of claim 1 or 2, wherein the humidifier and/or the heater of a patient conduit is configured to heat the gases flow to a desired temperature, wherein the desired temperature is: about equal to the desired dew point, above the desired dew point, about an offset plus the desired dew point, and/or based on the desired dew point.

5. The surgical humidification system of claim 4, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

6. A surgical humidification system, comprising:
a flow generator configured to control a gases flow to be provided to a surgical cavity at a desired flow rate, wherein the desired flow rate is a flow rate of about 3 litres/minute to about 120 litres/minute,
a humidifier configured to heat and humidify the gases flow to be provided to a surgical cavity,
wherein the humidifier and/or a heater of a patient conduit pneumatically connected to the humidifier, is configured to humidify the gases flow to a desired dew point of about 35 degrees Celsius to about 43 degrees Celsius, or about 40 degrees Celsius, and to heat the gases flow to a desired temperature about equal to the desired dew point, about an offset plus the desired dew point, above the desired dew point, and/or based on the desired dew point.

7. A surgical humidification system, comprising:
a flow generator configured to control a gases flow to be provided to a surgical cavity at a desired flow rate, wherein the desired flow rate is a flow rate of about 45 litres/minute to about 65 litres/minute,
a humidifier configured to heat and humidify the gases flow to be provided to a surgical cavity,
wherein the humidifier and/or a heater of a patient conduit connected to the humidifier is configured to humidify the gases flow to a desired dew point of about 38 degrees Celsius to about 41 degrees Celsius, and to heat the gases flow to a desired temperature of about 38 degrees Celsius to about 43 degrees Celsius, about equal to the desired dew point, about an offset plus the desired dew point, above the desired dew point, and/or based on the desired dew point.

8. The surgical humidification system of claim 7, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

9. The surgical humidification system of any preceding claim, wherein the desired flow rate is provided as a continuous flow rate.

10. The surgical humidification system of any preceding claim, wherein the humidifier has a separate heater and a separate humidification unit, wherein the heater is configured to heat the gases and the humidification unit is configured to humidify the gases.

11. The surgical humidification system of any preceding claim, wherein the dew point is measured at or near an outlet of the humidifier, at or near an end of an outlet of the humidifier, at or near an end of a conduit connected to the humidifier, at or near an inlet of a cannula which is configured to deliver the gases flow to the surgical site, at or near an outlet of a cannula which is configured to deliver the gases flow to the surgical site, or in the surgical cavity.

12. The surgical humidification system of any preceding claim, wherein the gases flow is provided to the surgical cavity at a temperature at or above the desired dew point.

13. The surgical humidification system of any preceding claim, wherein the desired dew point is at or about atmospheric pressure, or at or about atmospheric+the pressure of the surgical cavity, or at or about atmospheric+10mm Hg.

14. The surgical humidification system of any preceding claim, wherein the system is configured to control a dew point of the gases to the desired dew point.

15. The surgical humidification system of claim 14, wherein the dew point of the gases is based on the output of one or more sensors.

16. The surgical humidification system of claim 14 or 15, wherein the dew point of the gases is estimated based on at least the output of a at least one temperature sensor, and/or at least one flow rate sensor.

17. The surgical humidification system of any preceding claim, wherein the desired dew point is a dew point at a location downstream of the humidifier.

18. The surgical humidification system of any preceding claim, wherein the desired dew point is a dew point at a location:
a) at or near an inlet or outlet of a patient conduit,
b) at or near a patient end or a humidifier end of a patient conduit connected to the humidifier,
c) at or near a beginning of a conduit connected to the humidifier,
d) at or near an inlet of a cannula which is configured to deliver the gases flow to the surgical site,
e) at or near an outlet of a cannula which is configured to deliver the gases flow to the surgical site.
f) In the surgical cavity.

19. The surgical humidification system of claims 1-14, wherein the system is configured to control a temperature of the gases to the desired temperature.

20. The surgical humidification system of claim 19, wherein the temperature of the gases is based on the output of one or more sensors.

21. The surgical humidification system of any preceding claim, wherein the desired temperature is at a location:
a) at or near an inlet or outlet of a patient conduit,
b) at or near a patient end or a humidifier end of a patient conduit connected to the humidifier,
c) at or near a beginning of a conduit connected to the humidifier,
d) at or near an inlet of a cannula which is configured to deliver the gases flow to the surgical site,
e) at or near an outlet of a cannula which is configured to deliver the gases flow to the surgical site.
f) In the surgical cavity.

22. The surgical humidification system of any preceding claim, wherein the gases flow is Carbon Dioxide

23. The surgical humidification system of claim 1-21, wherein the gases flow comprises:
a) ambient air,
b) Oxygen,
c) Carbon Dioxide,
d) any combination of a)-c).

24. The surgical humidification system of any preceding claim, wherein the desired dew point and/or a desired gases temperature, and/or the desired flow rate are scaled based on a composition of the gases flow.

25. The surgical humidification system of any preceding claim, wherein the surgical cavity is a pneumoperitoneum of the patient.

26. The surgical humidification system of any preceding claim, wherein the gases flow is diffused as it enters the surgical cavity.

27. The surgical humidification system of any preceding claim, wherein the flow generator and humidifier are provided in a single housing.

28. The surgical humidification system of any preceding claim, wherein the flow generator is an insufflator.

29. The surgical humidification system of claims 1-28, wherein the flow generator comprises a blower.

30. The surgical humidification system of any preceding claim, wherein the flow generator is connected to a pressurised source of gases.

31. The surgical humidification system of claim 30, wherein the flow generator comprises a valve configured to provide the gases flow by controlling the outlet of the pressurised source of gases.

32. The surgical humidification system of any preceding claim, wherein the flow generator comprises a controller, and the humidifier comprises a controller.

33. The surgical humidification system of any one of claims 1-32, wherein an outlet of the humidifier is configured to pneumatically connect to a conduit, the conduit is configured to provide a passageway for the gases flow.

34. The surgical humidification system of claim 33, wherein the conduit comprises a heater.

35. The surgical humidification system of claim 34, wherein the heater of the conduit is configured to maintain the gases temperature at or above the desired dew point.

36. The surgical humidification system of claim 34 or 35, wherein the heater is a heater wire.

37. The surgical humidification system of claim 36, wherein the heater wire is located:
in a passageway of the conduit,
attached to a wall of the conduit,
embedded in a wall of the conduit.

38. The surgical humidification system of claims 35-37, wherein the conduit is connected to a cannula, the cannula configured to deliver the gases flow to the surgical cavity.

39. The surgical humidification system of any preceding claim, wherein the system comprises an exhaust line, the exhaust line providing a vent pathway from the surgical cavity.

40. The surgical humidification system of claim 39, wherein the exhaust line is connected to an exhaust cannula.

41. The surgical humidification system of claim 39 or 40, wherein the exhaust line is connected to a suction source.

42. The surgical humidification system of any preceding claim, wherein the system comprises at least one filter, where the filter is located upstream of the flow generator and/or downstream of the humidifier and/or between the flow generator and the humidifier.

43. The surgical humidification system of any preceding claim, wherein the system comprises at least one temperature sensor, the at least one temperature sensor configured to measure a temperature of the gases flow, wherein the at least one temperature sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

44. The surgical humidification system of any preceding claim, wherein the system comprises at least one flow rate sensor, the at least one flow rate sensor configured to measure a flow rate of the gases flow, wherein the at least one flow rate sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

45. The surgical humidification system of any preceding claim, wherein the system comprises at least one pressure sensor, the at least pressure sensor configured to measure a pressure of the gases flow, wherein the at least one pressure sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

46. The surgical humidification system of any preceding claim, wherein the system comprises at least one humidity sensor, the at least one humidity sensor configured to measure a humidity of the gases flow, wherein the at least one humidity sensor is located in a gases flow path:
a) at or near an end of an outlet and/or inlet of the humidifier,
b) at or near an end of a conduit connected to the humidifier,
c) at or near an end of a conduit connected to a patient interface,
d) at or near an inlet of a patient interface which is configured to deliver the gases flow to the surgical site,
e) at or near an outlet of a patient interface which is configured to deliver the gases flow to the surgical site,
f) in the flow generator,
g) any combination of a)-f).

47. The surgical humidification system of any preceding claim, wherein the gases flow is recirculated from the surgical cavity through the humidifier.

48. The surgical humidification system of claim 1-47, wherein the gases flow is recirculated from the surgical cavity through the humidifier by the flow generator as a recirculation flow generator.

49. The surgical humidification system of claim 48, wherein the recirculation flow generator is configured to provide gases at a or the desired flow rate.

50. The surgical humidification system of any preceding claim, wherein the gases flow is recirculated through a recirculation circuit, the recirculation circuit comprising a humidifier and the flow generator.

51. The surgical humidification system of any preceding claim, wherein the system comprises a primary flow generator (optionally an insufflator) configured to control the volume and/or pressure of the surgical cavity of the patient, and wherein the flow generator is a secondary flow generator.

52. The surgical humidification system of claim 51, wherein the primary flow generator (optionally the insufflator) is configured to provide a primary flow of gases to the surgical cavity of the patient.

53. The surgical humidification system of claim 52, wherein the primary flow generator (optionally the insufflator) is connected to an insufflation cannula, wherein the insufflation cannula provides the primary flow of gases to the surgical cavity of the patient.

54. The surgical humidification system of claim 51-53, wherein the system comprises a primary humidifier configured to humidify the primary flow of gases, and wherein the humidifier is a secondary humidifier.

55. The surgical humidification system of claim 54, wherein the primary flow generator (optionally the insufflator) and primary humidifier are provided in a single housing.

56. The surgical humidification system of any preceding claim, wherein the system comprises a gases recirculation circuit, the gases recirculation circuit comprising a recirculation flow generator as the flow generator, configured to recirculate gases from the surgical cavity of the patient through a humidifier and back to the surgical cavity of the patient.

57. A surgical humidification system, comprising:
a gases recirculation circuit, the gases recirculation circuit comprising a recirculation flow generator configured to recirculate gases from a surgical cavity of the patient through a humidifier and back to the surgical cavity of the patient.

58. The surgical humidification system of claim 56 or 57, wherein the recirculation flow generator is configured to recirculate the gases through the gases recirculation circuit.

59. The surgical humidification system of claims 56-58, wherein the humidifier is configured to humidify the recirculated gases in the recirculation circuit.

60. The surgical humidification system of claims 56-59, wherein the recirculation flow generator is configured to control the volume and/or pressure of the surgical cavity of the patient.

61. The surgical humidification system of claims 56-60, wherein the system comprises a primary flow generator, the primary flow generator is configured to control the volume and/or pressure of the surgical cavity of the patient and wherein the recirculation flow generator is a secondary flow generator.

62. The surgical humidification system of claims 56-61, wherein the primary flow generator is an insufflator to control a separate primary gases flow to be provided to the surgical cavity of a patient.

63. The surgical humidification system of claims 56-62, wherein the system comprises at least one recirculation cannula.

64. The surgical humidification system of claim 63, wherein the at least one recirculation cannula comprises a single recirculation cannula, the single recirculation cannula comprising an outlet gas pathway providing a pathway for gases from the surgical cavity and an inlet gas pathway providing a pathway for gases to the surgical cavity.

65. The surgical humidification system of claim 63 or 64, wherein at least one recirculation cannula comprises an inlet cannula providing an inlet gases pathway for gases to the surgical cavity and an outlet cannula providing an outlet gases pathway for gases from the surgical cavity.

66. The surgical humidification system of claim 65, wherein the system comprises a recirculation supply conduit configured to pneumatically connect the inlet gases pathway of the at least one recirculation cannula to an outlet of the humidifier.

67. The surgical humidification system of claim 65 or 66, wherein the system comprises a recirculation return conduit configured to pneumatically connect the outlet gases pathway of the at least one recirculation cannula to an inlet of the recirculation flow generator.

68. The surgical humidification system of claim 66 or 67, wherein the recirculation supply conduit and/or the recirculation return conduit comprise a heater.

69. The surgical humidification system of claim 68, wherein the heater is a heater wire

70. The surgical humidification system of claim 69, wherein the heater wire is located:
in a passageway of the conduit,
attached to a wall of the conduit,
embedded in a wall of the conduit.

71. The surgical humidification system of claims 56-70, wherein the system is configured to control at least one gases parameter of the gases flow to at least one first desired gases parameter if a patient heating criterion is not satisfied, and
wherein the system is operable to control the at least one gases parameter of the gases flow to at least one second desired gases parameter if a patient heating criterion is satisfied.

72. A surgical humidification system, comprising:
a flow generator configured to control a gases flow to be provided to a surgical cavity of a patient,
a humidifier, wherein the humidifier heats and/or humidifies the gases flow,
wherein the system is configured to control at least one gases parameter of the gases flow to at least one first desired gases parameter if a patient heating criterion is not satisfied, and
wherein the system is operable to control the at least one gases parameter of the gases flow to at least one second desired gases parameter if a patient heating criterion is satisfied.

73. The surgical humidification system of claim 71 or 72, wherein one or more of the at least one second desired gases parameter is lower than the corresponding at least one first desired gases parameter.

74. The surgical humidification system of claims 71-73, wherein the at least one gases parameter comprises any one or combination of: a dew point of the gases flow, a temperature of the gases flow, or a flow rate of the gases flow.

75. The surgical humidification system of claims 71-74, wherein the at least one first desired gases parameter comprises a first desired dew point and/or a first desired temperature and/or a first desired flow rate.

76. The surgical humidification system of claim 75, wherein the at least one second desired gases parameter comprises a second desired dew point and/or a second desired temperature and/or a second desired flow rate.

77. The surgical humidification system of claim 76, wherein the second desired dew point is lower than the first desired dew point.

78. The surgical humidification system of claim 76 or 77, wherein the second desired flow rate is lower than the first desired flow rate.

79. The surgical humidification system of claims 76-78, wherein the second desired temperature is lower than the first desired temperature.

80. The surgical humidification system of claims 75-79, wherein the first desired flow rate is about 40 litres/minute to about 55 litres/minute, or about 65 litres/minute.

81. The surgical humidification system of claims 75-80, wherein the first desired dew point is about 35 degrees Celsius to about 43 degrees Celsius, or about 38 degrees Celsius to about 41 degrees Celsius, or about 40 degrees Celsius.

82. The surgical humidification system of claims 75-81, wherein the first desired temperature is about equal to the first desired dew point, above the first desired dew point, about an offset plus the first desired dew point, and/or based on the first desired dew point.

83. The surgical humidification system of claims 75-82, wherein the first desired temperature is 38 degrees Celsius to about 43 degrees Celsius.

84. The surgical humidification system of claims 76-79, wherein the second desired flow rate is about 10 litres/minute to about 30 litres/minute, or about 10 litres/minute.

85. The surgical humidification system of claims 76-79 or 84, wherein the second desired flow rate is above a predetermined threshold.

86. The surgical humidification system of claim 85, wherein the predetermined threshold is 6 litres/minute, or about 10 litres/minute.

87. The surgical humidification system of claims 76-79 or 84-86, wherein the second desired dew point is about 33 degrees Celsius to about 37 degrees Celsius, or about 34 degrees Celsius, or about 37 degrees Celsius.

88. The surgical humidification system of claims 76-79 or 84-87, wherein the second desired temperature is about equal to the second desired dew point, above the second desired dew point, about an offset plus the second desired dew point, and/or based on the second desired dew point.

89. The surgical humidification system of claims 76-79 or 84-88, wherein the second desired temperature is 37 degrees Celsius to about 43 degrees Celsius, or above the desired dew point.

90. The surgical humidification system of claims 71-89, wherein the system is configured to determine that a patient does, or does not require heating is based on an input from a user.

91. The surgical humidification system of claim 90, wherein the input is from a user interface.

92. The surgical humidification system of claims 71-91, wherein the system is configured to determine that a patient does, or does not need heating based on the output of a patient temperature sensor.

93. The surgical humidification system of claim 92, wherein the patient temperature sensor is configured to measure a patient's core body temperature, or an external patient temperature.

94. The surgical humidification system of claim 93, wherein the system is configured to determine that a patient does not need heating when the patient's core body temperature and/or the external patient temperature is above a threshold.

95. The surgical humidification system of claim 93 or 94, wherein the system is configured to determine that a patient does need heating when the patient's core body temperature and/or the external patient temperature is below a threshold.

96. The surgical humidification system of claims 72-95, wherein the system comprises a gases recirculation circuit, the gases recirculation circuit comprising the flow generator as a recirculation flow generator configured to recirculate gases from the surgical cavity of the patient through the humidifier and back to the surgical cavity of the patient.

97. The surgical humidification system of claims 71-96, wherein the system comprises a venting cannula.

98. The surgical humidification system of claims 72-96, wherein the system comprises a heater of a patient conduit pneumatically connected to the humidifier configured to heat and/or humidify the gases flow in addition to the humidifier.

99. A method of transferring heat to a patient undergoing a surgical procedure to protect a patient from unintended perioperative hypothermia, the method comprising:
conditioning a gases flow, wherein conditioning the gases flow comprises humidifying the gases flow to a dew point higher than the patient's core body temperature and heating the gases flow to a temperature at or above the dew point, and
delivering the gases flow to a surgical cavity of a patient.

100. The method of claim 99, wherein conditioning the gases flow further comprises controlling the gases flow to a flow rate of about 3 litres/minute to about 120 litres/minute, or about 45 litres/minute to about 65 litres/minute.

101. The method of claim 99 or 100, wherein the dew point the gases are humidified to is about 35 degrees Celsius to about 43 degrees Celsius, or about 38 degrees Celsius to about 41 degrees

102. The method of claims 99-101, wherein the gases flow is heated to a desired temperature, wherein the desired temperature is: about equal to a desired dew point, above a/the desired dew point, about an offset plus a/the desired dew point, and/or based on a/the desired dew point.

103. The method of claim 102, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

104. The method of claims 99-103, wherein the gases flow is provided from a flow generator.

105. A method of transferring heat to a patient to protect a patient from unintended surgery-related hypothermia, the method comprising:
conditioning a gases flow, wherein conditioning the gases flow comprises humidifying the gases flow to a dew point higher than the patient's core body temperature and heating the gases flow to a temperature at or above the dew point, and
delivering the gases flow to a conduit.

106. The method of claim 105, wherein the conduit is suitable for inclusion in a cavity of a patient.

107. The method of claim 105 or 106, wherein the gases flow is delivered to the conduit at a flow rate of about 3 litres/minute to about 120 litres/minute, or about 45 litres/minute to about 65 litres/minute.

108. The method of claims 105-107, wherein the dew point the gases are humidified to is about 35 degrees Celsius to about 43 degrees Celsius, or about 38 degrees Celsius to about 41 degrees

109. The method of claims 1-5-108, wherein the gases flow is heated to a desired temperature, wherein the desired temperature is: about equal to a desired dew point, above a/the desired dew point, about an offset plus a/the desired dew point, and/or based on a/the desired dew point.

110. The method of claim 109, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

111. A method of conditioning a gas flow, the method comprising:
humidifying the gases flow to a dew point wherein the dew point is about 35 degrees Celsius to about 41 degrees Celsius, or about 38 degrees Celsius to about 41 degrees Celsius, and heating the gases flow to a temperature at or above the dew point.

112. The method of claim 111, wherein the gases flow is at a flow rate of about 3 litres/minute to about 120 litres/minute, or about 45 litres/minute to about 65 litres/minute.

113. The method of claim 111 or 112, wherein the dew point the gases are humidified to is about 35 degrees Celsius to about 43 degrees Celsius, or about 38 degrees Celsius to about 41 degrees

114. The method of claims 111-113, wherein the gases flow is heated to a desired temperature, wherein the desired temperature is: about equal to a desired dew point, above a/the desired dew point, about an offset plus a/the desired dew point, and/or based on a/the desired dew point.

115. The method of claim 114, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

116. The method of claims 111-115, wherein the method is provided by the system of any of claims 1 to 98.

117. A composition comprising carbon dioxide having a dew point of about 35 degrees Celsius to about 41 degrees Celsius, or about 38 degrees Celsius to about 43 degrees Celsius and at a temperature of about 38 degrees Celsius to about 41 degrees, an offset plus the dew point, or above the dew point, for use in a method of transferring heat to a patient undergoing a surgical procedure to protect a patient from unintended perioperative hypothermia, the method comprising delivering the composition to the surgical cavity of a patient.

118. The composition of claim 117, wherein the composition is delivered at a flow rate of 3 litres/minute to about 120 litres/minute, or about 45 litres/minute to about 65 litres/minute.

119. The composition of claim 117 or 118, wherein in addition to carbon dioxide the composition comprises ambient air and/or Oxygen.

120. A surgical humidification system, comprising:
a flow generator configured to control a gases flow to be provided to a surgical cavity,
a humidifier configured to humidify the gases flow to be provided to the surgical cavity,
wherein the gases flow is provided at a flow rate and humidity such that condensation of water vapour in the surgical cavity heats the patient during a surgical procedure.

121. The surgical humidification system of claim 120, wherein the flow rate is about 3 litres/minute to about 120 litres/minute, or about 45 litres/minute to about 65 litres/minute.

122. The surgical humidification system of claim 120 or 121, wherein the humidity is a dew point, and the gases are humidified to is about 35 degrees Celsius to about 43 degrees Celsius, or about 38 degrees Celsius to about 41 degrees.

123. The surgical humidification system of claims 120-122, wherein the gases flow is heated to a desired temperature, wherein the desired temperature is: about equal to a desired dew point, above a/the desired dew point, about an offset plus a/the desired dew point, and/or based on a/the desired dew point.

124. The surgical humidification system of claim 123, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

125. A surgical humidification system, comprising:
a flow generator configured to control a gases flow to be provided to a surgical cavity to a desired flow rate,
a humidifier, wherein the humidifier heats and humidifies the gases to a desired dew point,
wherein the system is configured to transfer energy to the patient, wherein the energy is provided by at least condensation of water vapour on tissue of the patient within the surgical cavity, wherein the energy transferred to the patient is based on at least: the desired flow rate and the desired dew point.

126. The surgical humidification system of claim 125, wherein the desired flow rate is about 3 litres/minute to about 120 litres/minute, or about 45 litres/minute to about 65 litres/minute.

127. The surgical humidification system of claim 125 or 126, wherein the desired dew point is about 35 degrees Celsius to about 43 degrees Celsius, or about 38 degrees Celsius to about 41 degrees

128. The surgical humidification system of claims 125-127, wherein the gases flow is heated to a desired temperature, wherein the desired temperature is: about equal to a desired dew point, above a/the desired dew point, about an offset plus a/the desired dew point, and/or based on a/the desired dew point.

129. The surgical humidification system of claim 128, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

130. A surgical humidification system, comprising:
a flow generator configured to control a gases flow to be provided to a surgical cavity,
a humidifier configured to humidify the gases flow to be provided to the surgical cavity,
a gases recirculation circuit, the gases recirculation circuit comprising a recirculation flow generator configured to recirculate gases from the surgical cavity of the patient through a humidifier and back to the surgical cavity of the patient,
wherein the gases flow is provided at a flow rate, humidity and/or temperature so as to reach or maintain normothermia for a patient.

131. The surgical humidification system of claim 130, wherein the desired flow rate is about 3 litres/minute to about 120 litres/minute, or about 45 litres/minute to about 65 litres/minute.

132. The surgical humidification system of claim 130 or 131, wherein the desired dew point is about 35 degrees Celsius to about 43 degrees Celsius, or about 38 degrees Celsius to about 41 degrees

133. The surgical humidification system of claims 130-132, wherein the gases flow is heated to a desired temperature, wherein the desired temperature is: about equal to a desired dew point, above a/the desired dew point, about an offset plus a/the desired dew point, and/or based on a/the desired dew point.

134. The surgical humidification system of claim 133, wherein the offset is about 1 degree Celsius to about 5 degrees Celsius, or about 1 degree Celsius to about 3 degrees Celsius, or about 1 degree Celsius to about 2 degrees Celsius, or about 1 degree Celsius, or about 2 degrees Celsius.

135. A surgical humidification system, comprising:
a flow generator configured to control a gases flow to be provided to a surgical cavity to a desired flow rate,
a humidifier, wherein the humidifier heats and humidifies the gases to a desired dew point,
wherein the system is configured to transfer energy to the patient, wherein the energy is provided by at least condensation of water vapour on tissue of the patient within the surgical cavity, wherein the energy transferred to the patient is based on at least: the desired flow rate and the desired dew point.

136. A method of providing a flow of gases to a surgical cavity, the method comprising:
monitoring a patient temperature,
conditioning the flow of gases according to one or more operational parameters, wherein the one or more operational parameters comprise at least the dew point of the flow of gases, wherein the dew point of the flow of gases is above the monitored patient temperature, and
delivering the gases flow to a surgical cavity.
